(19) United States
(12) Patent Application Publication
(10) Pub. No.: US 2012/0040461 A1
(43) Pub. Date: Feb. 16, 2012

(54) FABRICATION OF NANOFIBER REINFORCED STRUCTURES FOR TISSUE ENGINEERING

(76) Inventors: Vince Z. Beachley, Mt. Pleasant, SC (US); Xuejun Wen, Mt. Pleasant, SC (US)

(21) Appl. No.: 13/201,874

(22) PCT Filed: Feb. 23, 2010
(86) PCT No.: PCT/US10/24987

§ 371 Date: Oct. 31, 2011

Related U.S. Application Data
(60) Provisional application No. 61/154,550, filed on Feb. 23, 2009.

(51) Int. Cl. C12N 5/071; B29C 47/00; B32B 37/00; B32B 5/02

(52) U.S. Cl. 435/396; 428/74; 264/465; 156/62.4

(57) ABSTRACT

Disclosed are composite arrays and methods of forming the arrays. Composite arrays include a hydrogel-forming polymeric network and a network of electrospun fibers embedded within the polymeric network. For instance, the polymeric network can include one or more extracellular matrix proteins. The network of electrospun fibers can describe an open configuration that incorporates sufficient space between adjacent fibers to allow for cellular ingrowth between and among individual fibers. Disclosed composite arrays can be utilized as a supporting scaffold for living cells, for instance in development of bioengineered tissue constructs.

### CROSS REFERENCE TO RELATED APPLICATION

The present application claims filing benefit of U.S. Provisional patent application Ser. No. 61/154,550 having a filing date of Feb. 23, 2009 entitled “Fabrication of Nanofiber Reinforced Protein Structures For Tissue Engineering,” which is incorporated herein by reference in its entirety.

### BACKGROUND

The basic concept of electrostatic spinning (or electrospinning) a polymer to form extremely small diameter fibers was first patented by Anton Formhals (U.S. Pat. No. 1,975,504). Electrostatically spun fibers and nonwoven webs formed therefrom exhibit very high surface areas and can be formed from a wide variety of polymers and composites. Due in part to the extremely high surface area of electrospun nonwoven webs, these materials show promise for use in tissue engineering. Electrospun nonwoven webs formed by traditional methods include fibers laid down in a random orientation, which can be favorable for some tissue engineering applications. Aligned orientations are more favorable for other applications due to the capability of directional guidance during tissue growth and development.

Improvements to the basic electrospinning process have been developed over the years. FIG. 1 illustrates one method of electrospinning fibers in an aligned orientation. According to this process, parallel conductive silicon plates on either side of an air gap (FIG. 1A) produce an electric field (FIG. 1B) that aligns the deposited fibers across the air gap (FIG. 1C) (see, e.g., Li, et al., Nanoletters, 2003, 3:8, 1167, which is incorporated herein by reference). This method has been used to collect two dimensional arrays of aligned and oriented fibers.

Unfortunately, problems still exist with aligned fiber arrays formed according to the methods of FIG. 1, particularly when attempts have been made to utilize the materials in tissue engineering applications. For instance, as fibers are collected one on top of another, the newly formed, still wet fibers adhere and bond to adjacent fibers above and below within the web. As a result, the as-fabricated arrays are very dense with little porosity. These dense arrays are not suitable in bioengineering applications as scaffolding material, as the dense mats can only allow cells to grow on the surface, and development of a three dimensional cellular construct throughout the depth of the array is not possible.

Other materials have been examined for bioengineering applications. For example, attempts have been made to utilize extracellular matrix (ECM) polymers to form an environment more closely resembling the natural environment in which cells grow and develop. For instance, hydrogels formed of extracellular proteins such as collagen, gelatin, laminin, and the like, have been developed. Hydrogels are multi-phase systems that can include a dispersed aqueous phase distributed in a continuous stable polymeric phase. As such, a hydrogel can vary from a free flowing liquid to a rigid or semi-rigid solid depending upon, e.g., hydration level, pH, temperature, etc. ECM protein-based hydrogels can exhibit excellent biocompatibility and can allow cell in-growth and survival, but suffer from other failings, primarily structural weakness. The overall weakness of ECM gels formed to date makes them difficult to handle and greatly limits the usefulness of the materials. Specifically, structural weakness not only limits ECM proteins to utilization as coating materials or in non-load bearing environments, but also causes problems in handling and manipulation during fabrication, processing, and implantation steps.

What are needed in the art are improved arrays for supporting and encouraging the development of bioengineered tissues and methods for forming such arrays.

### SUMMARY

In one embodiment, disclosed is a biocompatible composite structure. For instance, a biocompatible composite structure can include a first array of biocompatible electrospun nanofibers. More specifically, adjacent nanofibers of the first array can be substantially aligned with one another and can define a space between one another. A composite structure can also include a film encapsulating the array of biocompatible electrospun nanofibers such that the film is continuous over the array and is continuous across the space between adjacent nanofibers of the array. In addition, the encapsulating film can include at least one biocompatible hydrogel-forming polymer, for instance an extracellular matrix protein.

Biocompatible composite structures as disclosed herein can be dried or hydrated. For instance, a structure can be dried during shaping, storage, shipping, etc., during which the film can be a dry film and can be rehydrated for utilization as a cellular scaffold upon which the film can be more gel-like in consistency.

A composite structure can be combined with additional layers to form a thicker structure. For instance, a structure can include a second array of biocompatible electrospun nanofibers that is layered with the first array. The second array can be external to the film, or can be encapsulated with the first array in the film. According to another embodiment, the first array can be encapsulated in a first film, and the second array can be encapsulated in a second film prior to layering the two together. Moreover, the two films can be the same or different, as desired. For instance, the second film can include different polymers or other components (e.g., crosslinking components) that can interact with a component of the first layer.

A composite structure can be further stabilized through the formation of bonds within and among layers of a structure. For instance, an extracellular matrix protein of an encapsulating film can be directly or indirectly bonded to another component of the composite structure such as another protein of the same or different type or a nanofiber.

Also disclosed are methods for forming a composite structure. By way of example, a method can include forming an array as described above and encapsulating the array in a film that includes at least one hydrogel-forming polymer.

In one preferred embodiment, an array of biocompatible electrospun nanofibers can be formed according to a method that includes electrospinning a first nanofiber from an electrospinning nozzle and depositing the first nanofiber at a deposition area. More specifically, the deposition area can be defined by a first collection surface and a second collection surface that is at a distance from the first collection surface such that upon formation the nanofiber is formed between the two surfaces. A method can then include moving the collection surfaces such that the first nanofiber is moved in a direction away from the deposition area. Following, a second nanofiber can be formed and deposited at the deposition area subsequent to the motion of the first nanofiber away from the deposition area. Accordingly, the two fibers will be substantially aligned with one another, and can define a space between one another.

### BRIEF DESCRIPTION OF THE FIGURES

A full and enabling disclosure, including the best mode thereof, to one of ordinary skill in the art, is set forth more particularly in the remainder of the specification, including reference to the accompanying figures, in which:

FIG. 1 is a schematic diagram of a prior art electrostatic spinning process used to form an aligned two dimensional nanofiber array.

FIG. 2 is a schematic diagram of one embodiment of a process as may be utilized to form an aligned nanofiber array.

FIGS. 3A and 3B are representations of another embodiment of a process as may be utilized to form an aligned nanofiber array.

FIGS. 4A-4F are photographs of one embodiment of a composite array formation process including collection of nanofibers (FIG. 4A), transfer of formed array to a smaller frame (FIG. 4B), separated smaller array (FIG. 4C), wetting of the formed array in a gelatin solution (FIG. 4D), removal of wet composite array from solution (FIG. 4E), and dried composite array (FIG. 4F).

FIGS. 5A-5F illustrate two composite arrays including a schematic of a unidirectional aligned nanofiber reinforced protein composite array (FIG. 5A), a schematic of a bidirectional nanofiber reinforced protein composite array (FIG. 5B), a fluorescent image of a composite array as illustrated in FIG. 5A (FIG. 5C), a fluorescent image of composite array as illustrated in FIG. 5B (FIG. 5D), an SEM image of a composite array as illustrated in FIG. 5A (FIG. 5E), and an SEM image of a composite array as illustrated in FIG. 5B (FIG. 5F).

FIG. 6A is a photograph of a composite array as disclosed herein mounted in a biaxial tester.

FIG. 6B graphically illustrates the force on both axes at 3% strain, 5% strain and 7% strain condition for a composite array including bidirectional nanofibers embedded in a protein matrix.

FIG. 6C graphically illustrates the force on both axes at 3% strain, 5% strain and 7% strain condition for a composite array including unidirectional nanofibers embedded in a protein matrix.

FIGS. 7A and 7B are photographs of a composite array as disclosed herein following handling and shaping.

FIGS. 8A-8D illustrate the elastic modulus of several different composite arrays as described herein tested under dry (FIG. 8A) and wet (FIG. 8B) conditions and failure stress of the same samples tested under dry (FIG. 8C) and wet (FIG. 8D) conditions.

FIGS. 9A and 9B illustrate 3T3 fibroblasts grown on composite arrays as described herein including bi-directional nanofiber orientations (FIG. 9A) and unidirectional nanofiber orientations (FIG. 9B).

FIG. 10 illustrates fibroblasts loaded onto a composite array including 1 mg/ml fibrinogen and 10 units/ml thrombin.

FIG. 11 illustrates fibroblasts loaded onto a composite array including 1 mg/ml fibrinogen and 20 units/ml thrombin.

FIG. 12 illustrates fibroblasts loaded onto a composite array including 1 mg/ml fibrinogen and 50 units/ml thrombin.

FIG. 13 illustrates fibroblasts loaded onto a composite array including 2.5 mg/ml fibrinogen and 10 units/ml thrombin.

FIG. 14 illustrates fibroblasts loaded onto a composite array including 2.5 mg/ml fibrinogen and 20 units/ml thrombin.

FIG. 15 illustrates fibroblasts loaded onto a composite array including 2.5 mg/ml fibrinogen and 50 units/ml thrombin.

FIG. 16 illustrates fibroblasts loaded onto a composite array including 5 mg/ml fibrinogen and 10 units/ml thrombin.

FIG. 17 illustrates fibroblasts loaded onto a composite array including 5 mg/ml fibrinogen and 20 units/ml thrombin.

FIG. 18 illustrates fibroblasts loaded onto a composite array including 5 mg/ml fibrinogen and 50 units/ml thrombin.

FIG. 19 illustrates fibroblasts loaded onto a hydrogel including 1 mg/ml fibrinogen and 20 units/ml thrombin.

FIG. 20 illustrates fibroblasts loaded onto a hydrogel including 2.5 mg/ml fibrinogen and 20 units/ml thrombin.

FIG. 21 illustrates fibroblasts loaded onto a hydrogel including 5 mg/ml fibrinogen and 20 units/ml thrombin.

FIG. 22 is a confocal image of a 2 day myoblast culture on a composite array including five sequentially formed composite electrospun fiber/protein layers each layer including 2.5 mg/ml fibrinogen.

FIG. 23 is another confocal image of a 5 day myoblast culture on a composite array including five sequentially formed composite electrospun fiber/protein layers each layer including 2.5 mg/ml fibrinogen.

FIG. 24 is a confocal image of a 2 day myoblast culture on a composite array including three sequentially formed composite electrospun fiber/protein layers each layer including 2.5 mg/ml fibrinogen.

FIG. 25 is another confocal image of a 2 day myoblast culture on a composite array including three sequentially formed composite electrospun fiber/protein layers each layer including 2.5 mg/ml fibrinogen.

FIGS. 26A-26C illustrate cryosection images of a multi-layer composite as disclosed herein.

Repeat use of reference characters in the present specification and drawings is intended to represent the same or analogous features or elements of the present invention.

### DETAILED DESCRIPTION

Reference will now be made in detail to various embodiments of the disclosed subject matter, one or more examples of which are set forth below. Each embodiment is provided by way of explanation, not limitation, of the subject matter. In fact, it will be apparent to those skilled in the art that various modifications and variations may be made in the present disclosure without departing from the scope or spirit of the disclosure. For instance, features illustrated or described as part of one embodiment, may be used in another embodiment to yield a still further embodiment. Thus, it is intended that the present subject matter cover such modifications and variations as come within the scope of the appended claims and their equivalents.

The present disclosure is generally directed to composite arrays and methods of forming the arrays. Composite arrays disclosed herein can show excellent characteristics for many biocompatible applications, and in one preferred embodiment, for use as support constructs in tissue engineering applications. More specifically, disclosed composite arrays include a hydrogel-forming polymeric network that can, in one embodiment include one or more extracellular matrix proteins. Additionally, a composite array can include a second network of electrospun fibers embedded within the hydrogel-forming polymeric network. Moreover, a network of electrospun fibers can be provided with an open configuration that incorporates sufficient space between adjacent fibers to allow for cellular ingrowth between and among individual fibers.

When utilized as a supporting scaffold for living cells, for instance in development of bioengineered tissue constructs, an electrospun fiber array can provide strength to the scaffold and directional guidance to developing cells, while a hydrogel-forming polymeric network can support cellular ingrowth between individual fibers, can improve biocompatibility of the composite array, and can provide a local environment for a cell that more closely resembles the natural in vivo environment in which that cell type can be found.

According to one preferred embodiment, electrospinning systems as disclosed in U.S. patent application Ser. No. 12/054,668, to Beachley, et al., which is incorporated herein by reference, can be utilized to form an electrospun nanofiber array. According to this embodiment, an electrospinning system can include a mobile fiber deposition location such that individual fibers can be moved away from the deposition location following formation and deposition of the fiber. The capability of forming loose fibrous constructs via such techniques can allow extra time for fibers to dry and avoid tight adhesion between fibers and can facilitate the fabrication of complex structures including electrospun fibers. For instance, formation processes can be utilized so as to provide fibrous constructs having controlled fiber packing density and allow for the optimization of void volume.

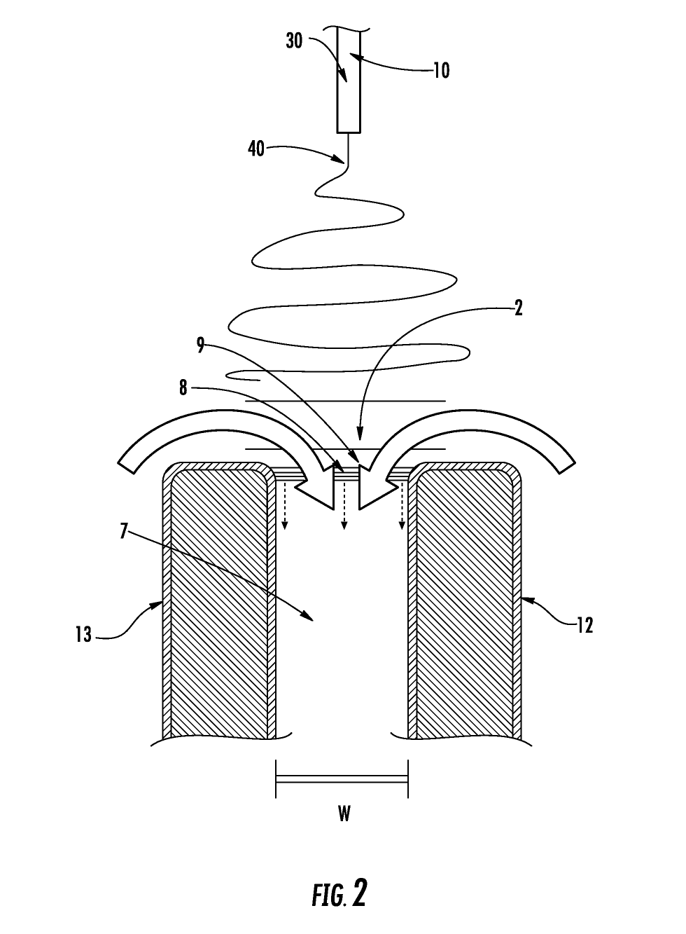

FIG. 2 is a schematic representation of one embodiment of an electrospinning process as may be utilized to form a fiber array. According to the illustrated process, an electrospinning nozzle 10 can be loaded with a polymeric composition 30. For instance, a biocompatible polymer, sol-gel, or composite solution or melt may be loaded into the electrospinning nozzle. Solutions and melts encompassed by the process can include homopolymers, block copolymers, random copolymers, polymeric blends, and so forth.

In general, a fiber array can be formed of any polymer that can be electrospun. In one embodiment, polymers for use in forming an electrospun fiber array can include biocompatible polymers. In one preferred embodiment, an electrospun fiber array can include biodegradable polymers. For instance, polymers that have been found suitable for use in biological applications such as alginates, polylactides, and the like can be utilized. In one embodiment, poly(hexano-6-lactone) (commonly referred to as ε-caprolactone or PCL) can be utilized. PCL is an aliphatic polyester that is a relatively stable synthetic polymer under usual conditions and is biodegradable under microbial attack. As such, PCL has garnered a great deal of interest for application in forming biodegradable plastics. In addition, PCL is biologically compatible and has been utilized extensively in forming tissue engineering scaffolds (see, e.g., U.S. Pat. No. 6,355,699 to Vyakarnam, et al., which is incorporated herein by reference).

Mixtures of materials can be electrospun in disclosed processes so as to form composite nanofibers, as is known in the art. For instance, a solution including one or more polymers in combination with a non-polymeric additive can be electrospun to form composite fibers. Additives can generally be selected based upon the desired application of the formed array. For example, one or more polymers can be electrospun with a biologically active additive that can be polymeric or non-polymeric, as desired. By way of example, an electrospun array can include a polymer in conjunction with one or more biologically active materials such as drugs, growth factors, nutrients, and the like. The secondary material can be incorporated in the fibers during formation as is known in the art, for example as described in U.S. Pat. Nos. 6,821,479 to Smith, et al., 6,753,454 to Smith, et al., and 6,743,273 to Chung, et al., all of which are incorporated herein by reference.

Individual fibers of an array can include polymeric and/or nonpolymeric materials in a random mixture throughout the fiber or in an ordered arrangement. For instance core/shell composite nanofibers can be formed with different materials forming the core and the shell. Formation methods for core/shell nanofibers have been described. For example, U.S. Patent Application Publication No. 2006/0226580 to Xia, et al., which is incorporated herein by reference, describes methods for forming core/shell and tubular nanofibers as may be utilized in a 3D electrospun fiber array.

A polymeric solution that is loaded into an electrospinning nozzle can include any suitable solvent. Selection of solvent can be of importance in determination of the characteristics of the solution, and hence of the characteristic properties of the nanofibers formed during the process. For instance, acetic acid, acetonitrile, m-cresole, tetrahydrofuran (THF), toluene, dichloromethane (CH₂Cl₂), methanol (MeOH), dimethylformamide, as well as mixtures of solvents are typical of solvents as may be utilized in disclosed processes.

As is generally known in the art, the critical field strength required to overcome the forces due to surface tension of the solution and form a jet will depend on many variables of the system. These variables include not only the type of polymer and solvent, but also the solution concentration and viscosity, as well as the temperature of the system. In general, characterization of the jet formed, and hence characterization of the fibers formed, depends primarily upon solution viscosity, net charge density carried by the electrospinning jet and surface tension of the solution. The ability to form the small diameter fibers depends upon the combination of all of the various parameters involved. For example, electrospinning of lower viscosity solutions will tend to form beaded fibers, rather than smooth fibers. In fact, many low viscosity solutions of low molecular weight polymers will break up into droplets or beads, rather than form fibers, when attempts are made to electrostatically spin the solution. Solutions having higher values of surface tension also tend to form beaded fibers or merely beads of polymer material, rather than smooth fibers. Thus, the preferred solvent for any particular embodiment will generally depend upon the other materials as well as the formation parameters, as is known in the art.

Referring again to FIG. 2, a polymeric composition 30, e.g., a solution or melt, can be loaded into an electrospinning nozzle 10. According to standard electrospinning methodology, upon application of a suitable voltage to the needle (generally on the order of about 5 to about 30 kV), the repulsive electrostatic forces induced at the liquid/air interface will overcome the surface tension forces, and a jet 40 of liquid will be ejected, as shown. The jet is first stretched into a Taylor cone structure. As the jet 40 travels toward the grounded deposition area, some of the solvent can evaporate, leaving behind charged polymer fibers 8, 9. As can be seen, the deposition area 2 can be between two spaced apart collection surfaces 12, 13. Accordingly, the charged polymer fibers 8, 9 can align in the air gap, i.e., in the deposition area 2, between the collection surfaces 12, 13, as illustrated, with either end of the fibers 8, 9 adhering to the respective collection surface 12, 13, as shown. In this particular embodiment, the deposition area 2 is near the top of a collection compartment 7.

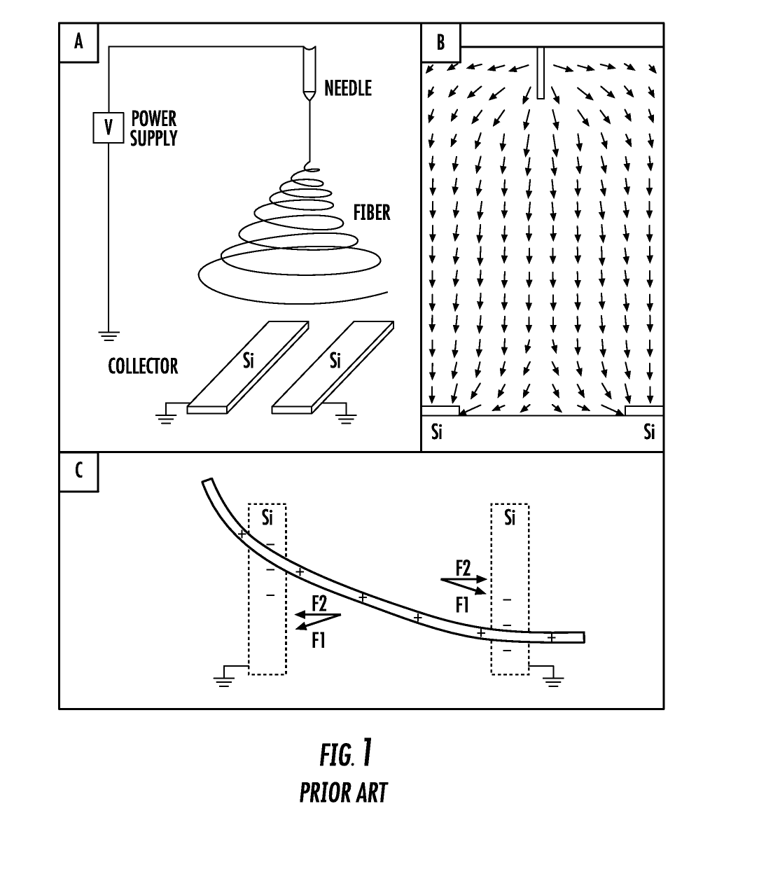

The relationship between the conductive collection surfaces 12, 13 and the applied voltage at the needle produces an electric field, similar to that of the static system illustrated in FIG. 1B, that aligns the nascent fibers 8, 9 and causes deposition of the fibers in generally parallel alignment across the gap between the two collection surfaces 12, 13 in the deposition area 2. Accordingly, the fibers can be substantially aligned with one another. As utilized herein, the term “substantially aligned” with regard to the nanofibers of an array includes perfectly aligned, i.e., parallel, fibers as well as fibers that are at a slight angle (e.g., less than about 30°) to one another along all or a portion of their length, such that two fibers may cross one another at some point, but both fibers terminate on the collection surfaces 12, 13 that are spaced apart from one another.

Materials as may be utilized in forming the collection surfaces 12, 13 can be any conductive material as is generally known in the art. For example, collection surfaces 12, 13 can be the same or different as one another and can include, without limitation, aluminum, copper, a laminate structure including a surface layer of a conductive material, or the like.

The system of FIG. 2 also includes the capability of mobility such that following deposition in the deposition area 2, the nascent fibers can be moved away from the deposition area 2. For instance, in the embodiment illustrated in FIG. 2, the collection surfaces 12, 13 of the system can be endless tracks formed of a conductive material that move as illustrated by the directional arrows in FIG. 2 and move the newly formed fibers away from the deposition area 2 and into collection compartment 7.

Surfaces 12, 13 can generally be separated from one another by a distance W of between about 2 mm up to about 10 cm, or even greater in other embodiments, for instance up to about 20 cm or even greater. In one embodiment, fibers of a length of up to about 50 cm can be formed. Maximum possible width, W, is generally understood to be related to fiber diameter, as well as other formation parameters. For instance, nanofibers can be formed with average diameters from about 350 nm to about 1 μm and having a length of between about 35 cm and about 50 cm. Accordingly, W of an array incorporating such fibers would likewise be between about 35 cm and about 50 cm. In another embodiment, W can be between about 2 cm and about 9 cm.

During formation, an individual fiber 8 can be deposited in the air gap between the collection surfaces 12, 13, as shown. Collection surfaces 12, 13 can rotate down through the collection compartment 7, and the newly formed fiber 8, which is adhered to the collection surfaces 12, 13 at either end of the fiber 8, can move down into the collection compartment 7 with the moving collection surfaces 12, 13. Beneficially, an individual fiber 8 can move away from deposition area 2 and down into collection compartment 7 prior to deposition of a second fiber 9 immediately above fiber 8. Thus, there can be space between the two fibers 8, 9. As such, remaining solvent on a fiber 8, 9 can dissipate and the fibers can dry while separated from one another such that the individual fibers that form a finished array need not be tightly adhered to one another.

The speed of the collection surfaces 12, 13 can control the vertical distance between fibers of the nascent array. For instance, surfaces 12, 13 can move at a speed of between about 1 cm/min and about 100 cm/min, for instance about 40 cm/min. Slower and faster speeds for the collections surfaces 12, 13 are possible in other embodiments. For instance, in another embodiment, collection surfaces 12, 13 can move at a speed of between about 0.5 cm/min and about 100 cm/min, or at speeds greater than 100 cm/min in other embodiments. Fiber density of the array can thus be controlled, as fiber density will increase with decreasing speed of the collection surfaces 12, 13. A system can define a minimum formation speed of collection surfaces 12, 13. At the minimum formation speed no new fibers will collect at deposition area 2 due to charge repulsion from the previously formed fibers. The minimum formation speed for any particular embodiment can depend upon the nature of the formed fibers, the width W of the array, the induced charge, and the like.

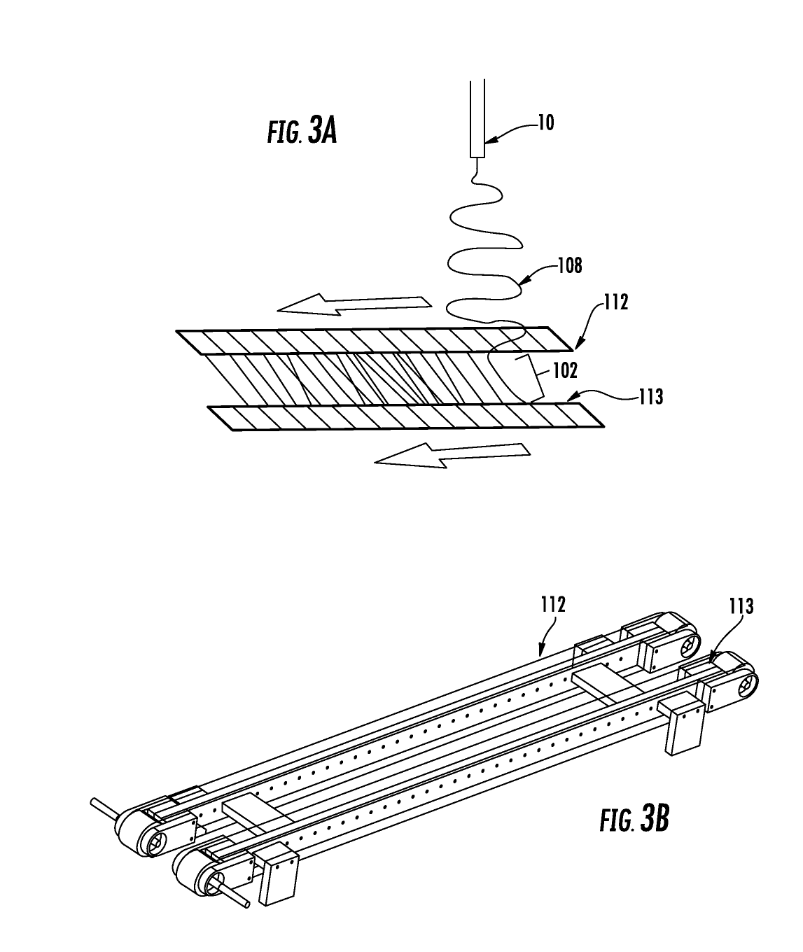

The motion of a deposited fiber away from the deposition area can be in any direction, and is not limited to the z-direction as defined by the electrospinning nozzle and as illustrated in FIG. 2, above. For instance, in another embodiment, illustrated in FIGS. 3A and 3B, following deposition at a deposition area 102 between collection surfaces 112, 113, a formed fiber 108 can move away from the deposition area 102 while remaining in the same plane of formation, as shown, i.e., in a direction normal to that direction defined by electrospinning jet. According to this embodiment, a thin, long array can be formed.

An electrospun array can also be versatile with respect to the materials used to form an array. For example, the polymeric composition ejected from the electrospinning nozzle can be varied throughout an array. According to one such embodiment, following formation of a first section of an array from a first polymer solution, the motion of collection surfaces 112, 113 can be stopped while a second, different polymer solution is loaded into the electrospinning nozzle 10, and then formation can resume.

Formation methods are not limited to utilization of a single ejection nozzle, and a plurality of nozzles can be utilized to form an electrospun array, if desired. Moreover different ejection nozzles can eject the same or different polymeric solutions. For instance, a first electrospinning nozzle can eject fibers from a first polymeric composition and a second, adjacent electrospinning nozzle can eject fibers from a second, different polymeric composition. Thus, a formed array can comprise different fibers at various locations throughout the array.

Different polymeric compositions can differ by any one or more components. For instance two different polymeric compositions can differ by solvent composition, which can lead to nanofibers being formed of identical materials, but having different diameters. In one embodiment, the polymers of two different nanofibers can be the same, and the nanofibers can differ by some secondary additive. Of course, the fibers can also differ from one another by one or more polymer components of the fibers. For instance, the microstructure of a polymer fiber can be varied through alteration of formation conditions such as solution characteristics including, viscosity and additives, through variation in applied voltage, and the like.

Referring again to FIG. 3, following loading of a different polymer solution in the electrospinning nozzle 10, motion of the collection surfaces 112, 113 can begin again as the second polymer solution is ejected from the nozzle 10. Thus, the nanofibers can differ by polymer, diameter, secondary additive, or the like through an array.

The overall size of an electrospun array can vary depending upon the final desired use. For instance, in addition to the variations in length and width, discussed above, an array can also vary in depth. A relatively thin electrospun array, for instance less than about 5 nanofibers in depth, can be preferred in one embodiment as the direction of the embedded electrospun fibers can be better sensed by cells that have attached and are developing on a scaffold.

In one embodiment an electrospun fibrous array can be formed including adjacent layers that are not aligned with one another. According to one such embodiment, a deposition area can be rotated during formation of the array so as to vary the orientation of the fibers throughout the depth and/or length of the array. For instance, a bidirectional array can be formed in which the fibers of adjacent layers are oriented at an approximately 90° angle to one another. Of course, the orientation of fibers of adjacent layers can be other than 90° as well. Moreover, as fiber orientation of layer(s) of the electrospun array can be utilized to direct growth and development of a developing tissue construct, this characteristic can be varied depending upon the desired cellular structure of a product tissue to be grown on the composite scaffold material.

Fibers of an array can include functionalization to promote bonding between the fibers and/or constituents of an encapsulating network. By way of example, fibers can be formed of materials that include or can be functionalized to include a reactivity moiety such as amine, carboxylate, or thio groups, that can allow covalent, non-covalent, charge/charge interaction, or any other type of bonding of a protein or other component of an encapsulating network to a fiber surface.

Surface chemistry modification of a fiber can be carried out to encourage bonding of a compound to a fiber. Fiber surface chemistry modification processes can include, without limitation, alkaline treatments, plasma treatments, corona discharge, and the like. Fiber surface modification can encourage bonding of a compound to the surface via any means. For example, fiber surface modification can increase the number of carboxylate groups on a fiber surface available for bonding to a protein of an encapsulating network.

Following formation, an electrospun array can be embedded in a network comprising one or more hydrogel-forming polymers, and in one embodiment, one or more ECM proteins. Preferred proteins to be included in a network can generally depend at least in part upon the cells to be developed on a finished scaffold. For instance, the primary protein of the ECM of plants is cellulose, while that of fungi, arthropods, and the like is chitin. When considering the growth and development of vertebrate cells, a proteinaceous network can include one or more collagens, laminins, elastins, fibronectin, and combinations thereof. In one preferred embodiment, a protein network can be a gelatin composition.

Proteins can be obtained from any suitable source. For instance, proteins can be natural proteins, obtained from the same organism type as the cell types to be developed on a composite array, or can be obtained from a different organism, or can be synthetic proteins, formed according to any suitable methodology. According to one embodiment, recombinant proteins can be utilized in forming a network. For example, in growth and development of human cells, a network formed of recombinant human proteins can be utilized, which can limit concerns with regard to immunogenicity and other issues, for instance for in vivo applications involving implantation of disclosed composite arrays.

Encapsulating networks are not limited to those formed of ECM proteins, however. Any biocompatible hydrogel-forming polymer can be utilized in forming a network. By way of example, the polymeric component of an encapsulating network can be formed of or can include polymers and/or copolymers of biocompatible materials that are not natural to the ECM such as a polyethylene glycol (PEG), a polylactic acid (PLA), polycaprolactones (PCL), a poly(glycolide) (PGA)).

Moreover, an encapsulating network can include ECM polymers other than or in addition to ECM proteins. For instance, a polymeric network can incorporate common ECM proteoglycans such as chondroitin sulfate, heparin sulfate, keratin sulfate, hyaluronic acid, and the like. In one preferred embodiment, the composition of an encapsulating network can be designed to closely match the natural environment of the cell types that will be loaded onto the composite scaffold material. In this embodiment, an encapsulating network can include multiple different ECM polymers including proteins, proteoglycans, and the like as well as other non-polymeric components of the ECM, for instance as a component of the aqueous portion of a hydrogel encapsulation.

An aqueous solution including the desired polymers and any other components can be formed and optionally processed to promote encapsulation of a fibrous array. For instance, a polymer solution that is a gel at ambient temperatures can be heated to form a liquid that can be applied as such to a fibrous array. In one embodiment, a formed electrospun array can be immersed in a heated proteinaceous solution. A polymer solution can be heated to a temperature that liquefies the solution without damaging the polymers, e.g., the proteins, of the solution. For example, a collagen-based solution can be heated to a temperature of between about 50° C. and about 65° C., which can liquefy the solution that is a gel at room temperature without degrading the collagen of the solution. Even at elevated temperature, the viscosity of a polymeric solution can be such that the solution can be applied to the electrospun array and form a continuous film encapsulating the array. More specifically, the surface tension of the polymeric solution can lead to the development of a continuous film covering the electrospun fibers of the array and extending unbroken across the open spaces between the electrospun fibers of the array.

Other application methods can alternatively be utilized. For instance, a solution can be poured over an electrospun array, can be sprayed onto an electrospun array, or any other application method that encourages the encapsulation of the fibrous array by the solution such that a unitary film encapsulates the electrospun array, i.e., the encapsulation of the array is not in the form of a simple continuous coating over the fibers, but rather a unitary film within which the array fibers are encapsulated and fills the spaces between adjacent fibers of the electrospun array.

The film encapsulating the electrospun array can vary in characteristics depending upon the specific polymers included in the film, the solids content of the solution, and the like. For instance, upon cooling from an increased application temperature a proteinaceous solution can thicken and form a semi-solid colloid hydrogel encapsulating the fibers of the electrospun array.

The volume fraction of components of a formed composite array can be varied depending upon desired characteristics of the formed array. For instance, arrays exhibiting excellent strength characteristics and good cellular ingrowth capabilities can be formed with nanofibers embedded in a hydrogel-forming network at a volume fraction of about 3% v/v. Volume fraction of composite array components can be readily adjusted by controlling the nanofiber fabrication conditions and collecting time. For example, a composite array including a higher volume fraction of electrospun fibers can be formed through either increasing the density of the fibers in the electrospun array (e.g., through decrease in speed of the collection surfaces or increase in collection time) or increasing the size of fibers of the array. A higher volume fraction of fibers can further increase strength characteristics of a formed composite array.

Additional processing of a composite array can be carried out. For instance, components of a composite array can be crosslinked. Suitable crosslinkers for polymer networks are generally known in the art, any of which may be utilized. For example, following encapsulation of a fibrous electrospun array by a proteinaceous solution, the composite array can be immersed in a solution including a suitable cross linking agent (e.g., genepin, thrombin, 1-ethyl-e-(3-dimethylaminopropyl) carbodiimide, and the like) that can crosslink and strengthen the composite array. A crosslinking agent can also be provided in the solution that encapsulates the electrospun array, rather than in a separate application step.

Crosslinking can be initiated, when desired, according to any standard methodology. For example, crosslinking can be induced via photo-initiation, temperature dependent crosslinking, and/or chemically activated crosslinking. For example, in one embodiment, proteins can be crosslinked to other proteins of the network or to fibers of the electrospun array in the presence of a photo-initiated crosslinking agent such as Irgacure® or Darocur® photoinitiators available from Ciba Specialty Chemicals. In another embodiment, a cationic initiator can be present. For example, a polyvalent elemental cation such as Ca²⁺, Mg²⁺, Al³⁺, La³⁺, or Mn²⁺ can be used. In another embodiment, a polycationic polypeptide such as polylysine or polyarginine can be utilized as crosslinker.

Crosslinking agents can form bonds between and among components of the encapsulating network as well as between components of the network and fibers of the electrospun array. For example, proteins can be crosslinked by using carboxy-activating crosslinking agents such as water-soluble carbodiimides. Such cross-linking agents can be used to crosslink proteins to one another, to cross link other macromolecules of a network to proteins, or to crosslink materials of the network to the fibers of the electrospun array. One example of this approach includes formation of a carbodiimide linkage of collagen or laminin with polylysine. Other hydroxylated entities can be linked in a similar manner. For example, in one embodiment, polyvinyl alcohol of a fibrous array can be linked with proteins using an epoxy-activation approach or crosslinked via polymerizable methacrylate groups along the side chains.

A hydrated composite array can be dried to form a composite including a dried polymer network encapsulating a fibrous array. The depth of a composite array can be relatively small, for instance, less than about 10 μm, or less than about 5 μm, in another embodiment. In one embodiment, a composite array can describe a relatively consistent thickness across the entire array of between about 2 μm and about 4 μm. Upon rehydration, the hydrogel characteristics of the array can recur and the array can again be suitable for use as a cellular scaffold. Beneficially, and due to the increased strength characteristics of the composite arrays, a composite array in the form of a dried film can be cut, shaped, and/or combined with other composite arrays as desired. For example, complex two and three-dimensional structures can be assembled from dry composite array films. Upon rehydration of the films, the structures can be suitable for use as cellular scaffolds.

Additional layers can be formed on a composite array, for instance in order to increase the overall depth of a cellular scaffold. For example, an additional layer of an electrospun array can be applied to one or both surfaces of a previously formed composite array, and polymer solution can be applied to this new array, thereby encapsulating the additional layer(s) of electrospun materials. Thus, the depth of the composite array can be increased. The process can be repeated as many times as desired to form a composite array of the desired depth. Additionally, adjacent fibrous layers can be encapsulated in the same or different hydrogel-forming solution. For example, a first fibrous array can be encapsulated in a first solution, and a second fibrous array can be encapsulated in a second solution following which the two layers can be combined to form a multi-layer composite structure. The two solutions can interact with one another following combination. For instance, two proteins of the two solutions can be crosslinked to one another following combination, so as to further strengthen the composite.

Cells can be loaded onto a composite array for culturing, development of a tissue construct, and the like. In one embodiment, cells can be loaded during formation of the composite array. For instance, a proteinaceous solution within which an electrospun array is to be embedded can include one or more cell types. Upon encapsulation of the array within the solution, the cells can be incorporated into the formed composite array. Alternatively, cells can be loaded onto an array following formation and any post-formation processing (e.g., drying, shaping, rehydrating, etc.). For example, a composite array can be loaded with cells through absorption and cellular migration, often coupled with application of pressure through simple stirring, pulsatile perfusion methods or application of centrifugal force.

Composite arrays as disclosed herein can be biocompatible as well as biodegradable cellular scaffolds that can promote cell attachment, survival and growth. Moreover, disclosed composite arrays can provide directional guidance to developing tissue constructs and posses excellent structurally stability for practical handling.

The disclosed subject matter may be better understood with reference to the following examples.

### Example 1

Polycaprolactone (PCL, Mn=80,000, Sigma, St Louis, Mo.) was dissolved in dichloromethane and dimethylformamide (3:1) (DCM:DMF, Sigma) at a concentration of 18% wt/v. A hydrophobic cyanine dye, 1,1′-dioctadecyl-3,3,3′3′-tetramethylindocarbocyanine perchlorate (Dil, Invitrogen, Carlsbad, Calif., USA), was added to the polymer solution at concentrations of 0.025 mg/ml to label the fabricated nanofibers. Polymer solution was fed by syringe pump (Medfusion 20101; Smiths Medial Inc., Carlsbad, Calif., USA) at a rate of 0.015 ml/min through a 23 G blunt tipped needle. A voltage of 8 KV was applied to the needle tip with a high voltage power supply (ES40P-IOW, Gamma High Voltage Research, Ormond Beach, Fla., USA). The needle tip was held at 10 cm above a collecting device as illustrated in FIG. 2. This device utilized parallel mobile tracks that pulled aligned electrospun fiber arrays into a secondary collection area at a vertical speed of 16.9 mm/s.

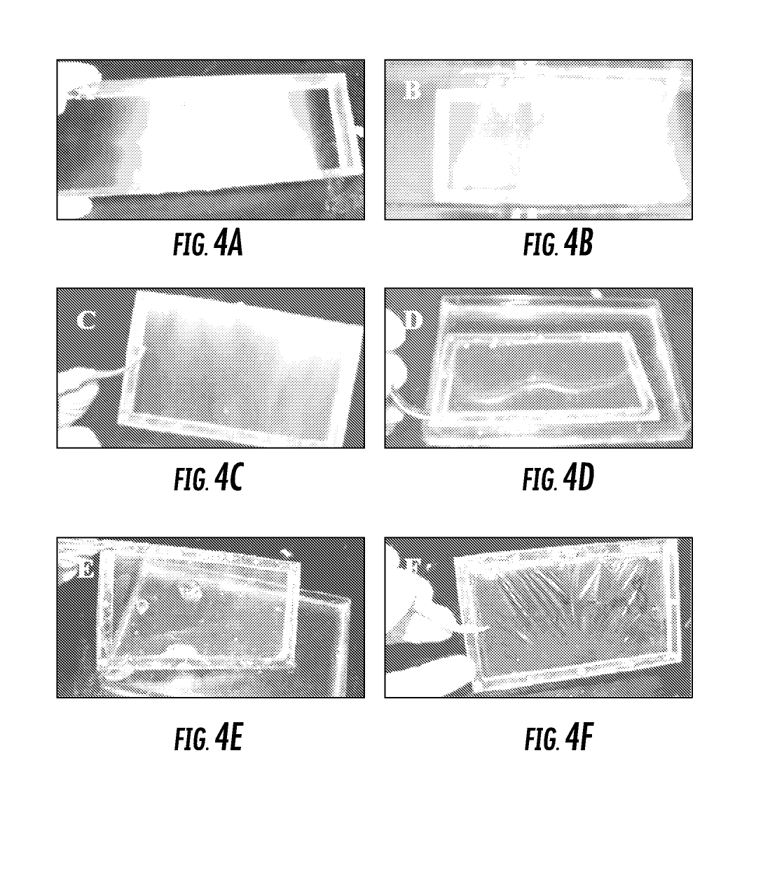

Parallel electrospun nanofibers were collected continuously across a rectangular rack (FIG. 4A) for 3 and 6 minutes, respectively, in different runs. The formed fiber array was then transferred to a two-piece polycarbonate frame that was held together with screws (FIGS. 4B and 4C).

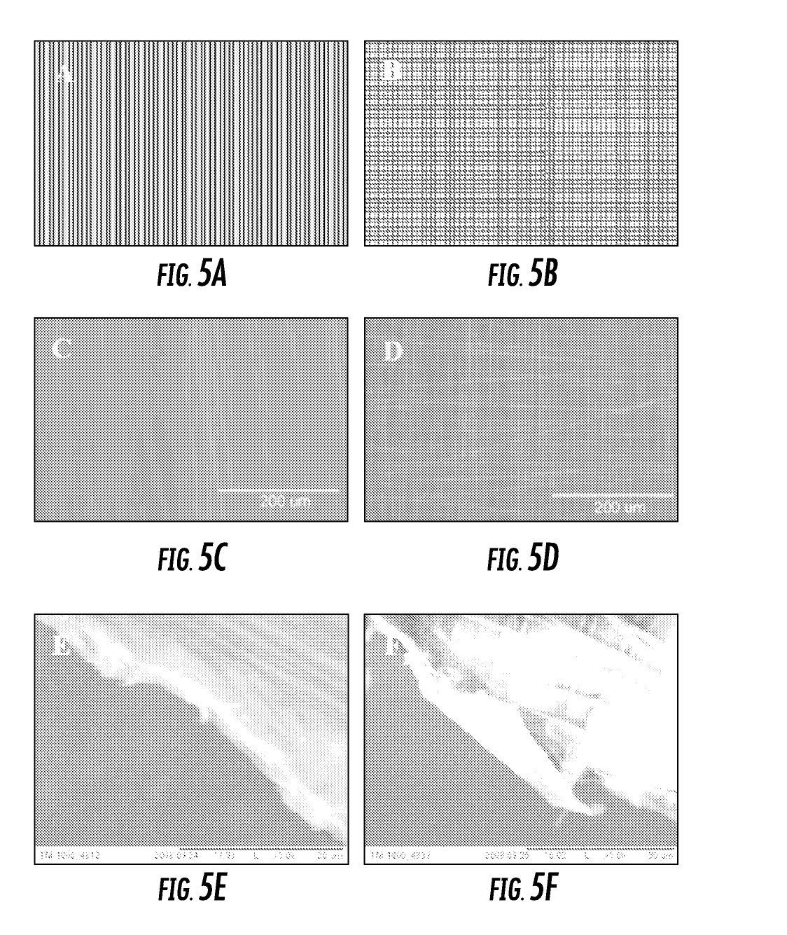

Composite arrays were fabricated with two different fiber orientations: unidirectional straight fibers (FIGS. 5A, 5C, and 5E) and bi-directional fibers (FIGS. 5B, 5D, and 5F).

During the fabrication of the composite arrays including unidirectional fibers, nanofibers were collected for 6 minutes and then transferred to a polycarbonate frame, as shown in FIG. 4C.

For fabrication of bi-directional composites, nanofibers were collected on a device as shown in FIG. 2 for three minutes. Following, these fibers were transferred to a rack as shown in FIG. 4B. A second array was then collected with a device as shown in FIG. 2. The square rack holding the first array was rotated 90 degrees and the second aligned array was put on top of the first array to form a single array including bi-directional fiber sets that were at approximately 90° to one another.

For each frame, one holding unidirectional fibers, and another holding bi-directional fibers, the frame was then immersed in a 1 wt % gelatin aqueous solution (Bovine skin type B, Sigma, St Louis, Mo., USA) (FIG. 4D). The materials were heated to approximately 50-60° C. during the immersion.

Several examples of each fiber orientation were formed. Following removal from the gelatin solution, some of the formed composites were cross-linked with 1-Ethyl-3-(3-dimethylaminopropyl) carbodiimide (EDC. TCI America) by immersion in a bath (either 75% ethanol or 66% acetone) with EDC. All the films were sterilized with 75% ethanol and allowed to dry. An example of a dried composite array is shown in FIG. 4F.

Fluorescent imagery and SEM confirmed that the nanofibers remained embedded in the gelatin films in their original configurations (FIGS. 5C and 5D are fluorescent images; FIGS. 5E and 5F are corresponding SEM images). All SEM images were taken at 1,000 to 5,000 times magnification using a scanning electron microscope (SEM, Hitachi TM-1000). Samples were not sputter coated. In order to visualize the embedded fibers, the edges of the composite arrays were selected for taking pictures. All fluorescent and light microscope images were taken using a Nikon Eclipse TE2000-S microscope with an EXFO X-cite 120 fluorescence illumination system—a Q-Imaging Micropublisher 3.3 RTV camera with Q-Capture software. FIGS. 5C-5F clearly show the fibers of each array that have been formed so as to be substantially aligned with one another. Additionally, the spaces between adjacent fibers of the arrays can also be seen in these images.

ImagePro Plus 4.0 (Media Cybernetics Inc., Bethesda, Md., USA) was used to measure the average thickness of composite array samples using SEM images of the cross sections (FIGS. 5E and 5F). The average thickness was found to be 2.73 μm with a standard deviation of 0.28 μm.

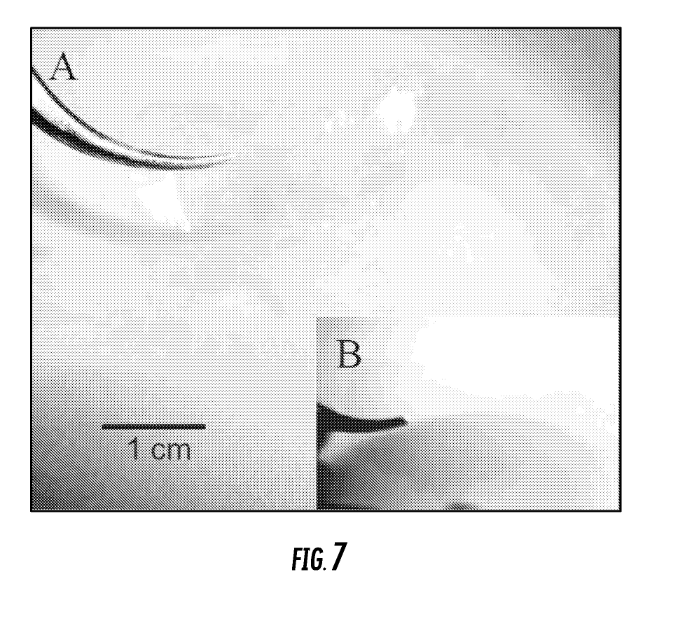

It was estimated from previously collected data that PCL fibers were approximately 585 nm in diameter and collected at a rate of approximately 0.05 fibers/μm/min (data not shown), therefore the estimated volume fraction of PCL nanofibers embedded in the composite films was about 3% v/v. Fabricated composite arrays were found to have the strength and structural integrity for manipulation into complex shapes. For instance, as shown in FIG. 7, a composite array was manipulated and cut to the shape of the United States, with no damage to the material.

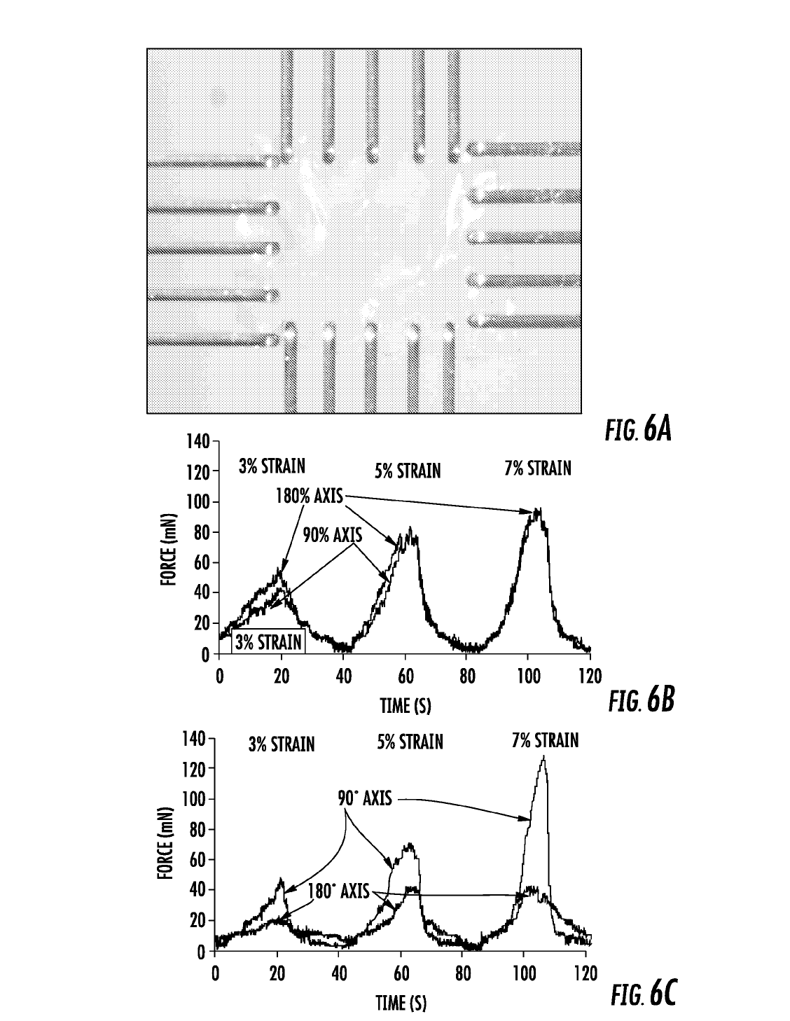

Mechanical Properties of the composite PCL nanofiber reinforced gelatin films were characterized using both biaxial tensile testing and uniaxial tensile testing. Biaxial tensile testing was performed using a BioTester5000 Biaxial Tester (CellScale division of Waterloo Instruments Inc., Waterloo, Ontario, Canada) in dry conditions. A typical set up for the biaxial tensile testing is shown in FIG. 6A. In brief, a dry composite array (5 mm×5 mm) was attached with 20 tungsten finger-grips (300 μm diameter) and stretched in the nanofiber direction or perpendicular to the nanofiber direction in dry condition. Each set of five finger grips was attached to an actuator, and load cells measured the force along the two pulling axes. The load cell used was 2500 mN. The samples were sequentially tested with 3% strain, 5% stain and 7% strain and each displacement was done in 10 seconds. Results are shown in FIGS. 6B and 6C. As can be seen, composite membranes reinforced with PCL nanofibers in a bi-directional pattern possessed similar mechanical properties in both 90° and 180° directions (FIG. 6B). In contrast, membranes with PCL nanofibers in a unidirectional pattern had significant differences in strength in different directions (FIG. 6C).

Uniaxial tensile testing was performed in a Shimadzu EZ Graph tensile tester (Nakagyo-ku, Kyoto, Japan) with Trapezium 2.32 software for data acquisition to examine the strength and stiffness of the thin films. 20 mm×5 mm rectangular strips of dry composite arrays were attached to the test grips. To limit stress concentrations on the thin films and prevent slippage from the grips, double sided SEM tapes were used to pad the ends of the film at the grip hold site. The length of the sample between two grips before testing was kept at 10 mm. A total of 20 samples were fabricated for mechanical testing, with 5 samples for each of 4 groups: (1) Unidirectional (2) Bi-directional (3) Unidirectional+EDC and (4) Bi-directional+EDC.

Strips were cut in the direction of the aligned fibers (90°) and perpendicular to the direction of the aligned fibers (180°). Strips cut from each sample were tensile tested in both directions under both wet and dry conditions. Under wet conditions the samples were secured in the grips of the tensile tester and then hydrated. The elastic modulus, maximum stress, and elongation were measured from the stress strain curves. Statistical analysis was done using the Mann-Whitney test with SPSS 15.0 software (SPSS Inc. Chicago, Ill., USA).

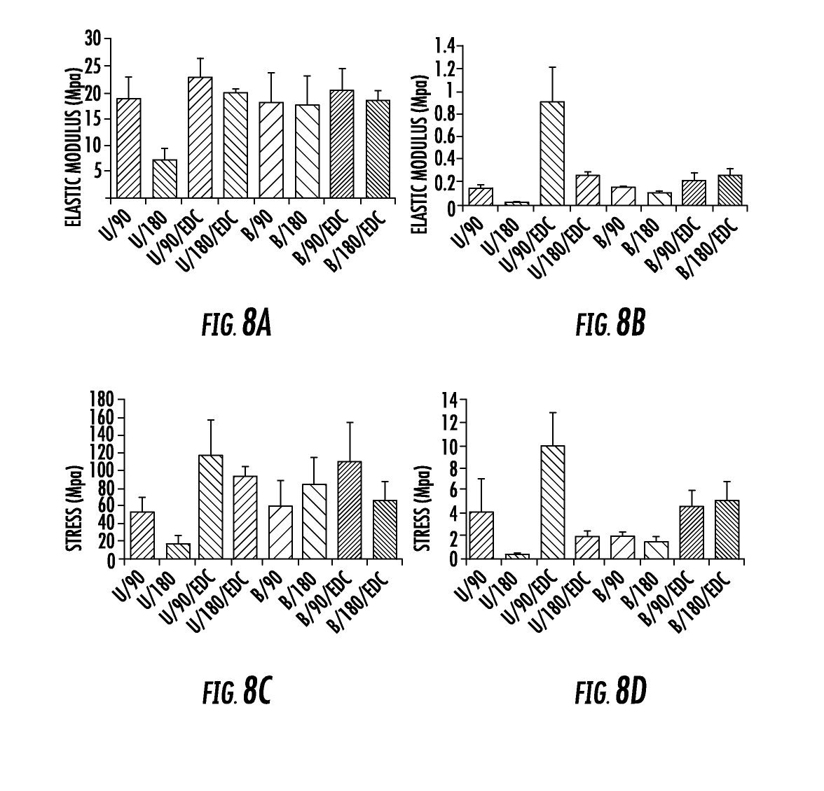

The elastic modulus and failure strength for each set of conditions is displayed in FIG. 8. Specifically, FIG. 8A illustrates elastic modulus under dry conditions, FIG. 8B illustrates elastic modulus under wet conditions, FIG. 8C illustrates failure strength under dry conditions, and FIG. 8D illustrates failure strength under wet conditions. As can be seen, fiber orientation had significant effects on the strength of the composite arrays for unidirectional samples both with and without EDC in both the dry and hydrated state. The elastic moduli of unidirectional composite films were 392% (p=0.05), 231% (p=0.02), and 164% (p=0.03) higher in the direction of the aligned fibers for hydrated, hydrated+EDC, and dry films respectively. The failure strength of unidirectional composite films were 1265% (p=0.05), 437% (p=0.03), and 212% (p=0.02) higher in the direction of the aligned fibers for hydrated, hydrated+EDC, and dry films respectively. Composite arrays cross-linked with EDC were stronger than films without EDC cross-linking over all fiber orientations (p<0.05). The elastic modulus of cross-linked films were 36% (p=0.02) higher in the dehydrated state, and 306% (p<0.01) higher when hydrated. The failure strength of cross-linked films were 86% (p<0.01) higher in the dehydrated state, and 139% (p=0.01) higher when hydrated.

Circular pieces of unidirectional and bi-directional EDC crosslinked composite film with a diameter of 19 mm were cut and placed at the bottom of 12 well cell culture plates. Half-inch sections of polycarbonate rod (Outer Diameter: 19 mm, Inner Diameter: 13 mm) were placed on top of the films to hold them down. Sample wells were sterilized with 75% ethanol, and 80,000 3T3 fibroblast cells were seeded on each sample and allowed to incubate for 3 days. Cells were fixed in 4% paraformaldehyde and stained with AlexaFluor 488 Phalloidin (Invitrogen) for the actin filament inside the cells and 4′-6-Diamidino-2-phenylindole (DAPI, Invitrogen) for the cell nuclei.

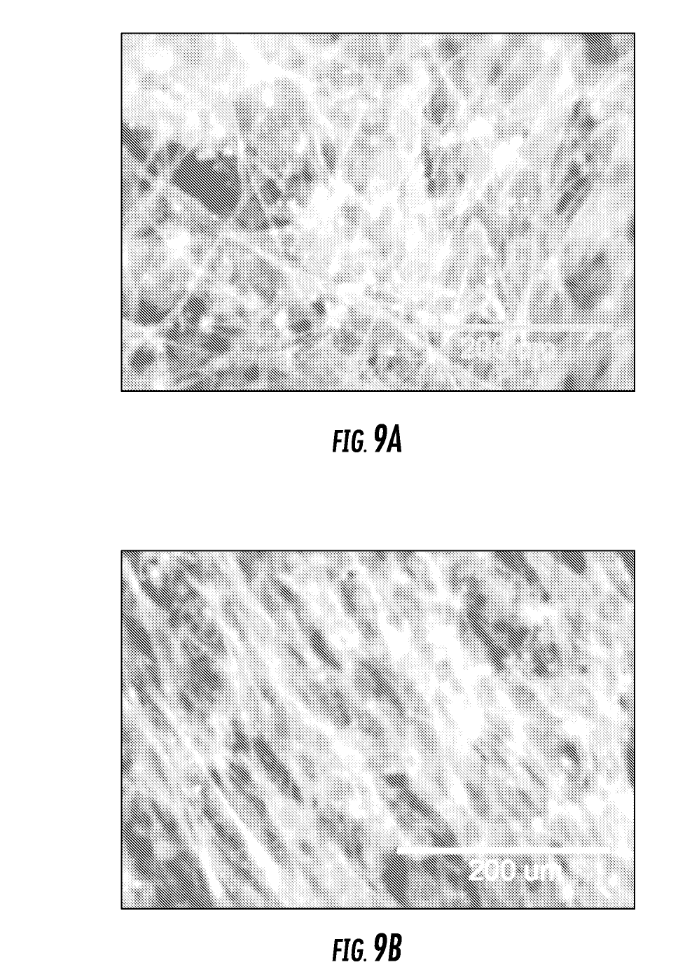

The composite arrays maintained their integrity under cell culture conditions and promoted the attachment of 3T3 fibroblast cells. Since the arrays were very thin, it is believed that cells were able to sense the embedded PCL nanofibers. Fibroblast cells cultured on the composite arrays incorporating bi-directional nanofibers are shown in FIG. 9A. Fibroblast cells cultured on the composite arrays incorporating unidirectional nanofibers elongated in the direction of the aligned nanofibers (FIG. 9B).

### Example 2

Aligned composite fiber arrays as described above in Example 1 were formed. The formed arrays were immersed into solutions including Bovine Fibrinogen (obtained from MP Biomedicals) in PBS. Various solutions were formed including either 1, 2.5, or 5 mg/ml fibrinogen. The wet films were then immediately immersed into solutions including thrombin as a crosslinking agent (available from Bovine MP Biomedicals) for 30 seconds. Crosslinking solutions including thrombin at either 10, 20, or 50 Units/ml were utilized. Each of three arrays including a single fibrinogen concentration was crosslinked with each of the different fibrin concentrations. Thus, nine different loaded composite fiber arrays were formed. As control, three systems including fibrinogen at 1, 2.5, and 5 mg/ml with thrombin were formed in wells without any electrospun fibers in the system.

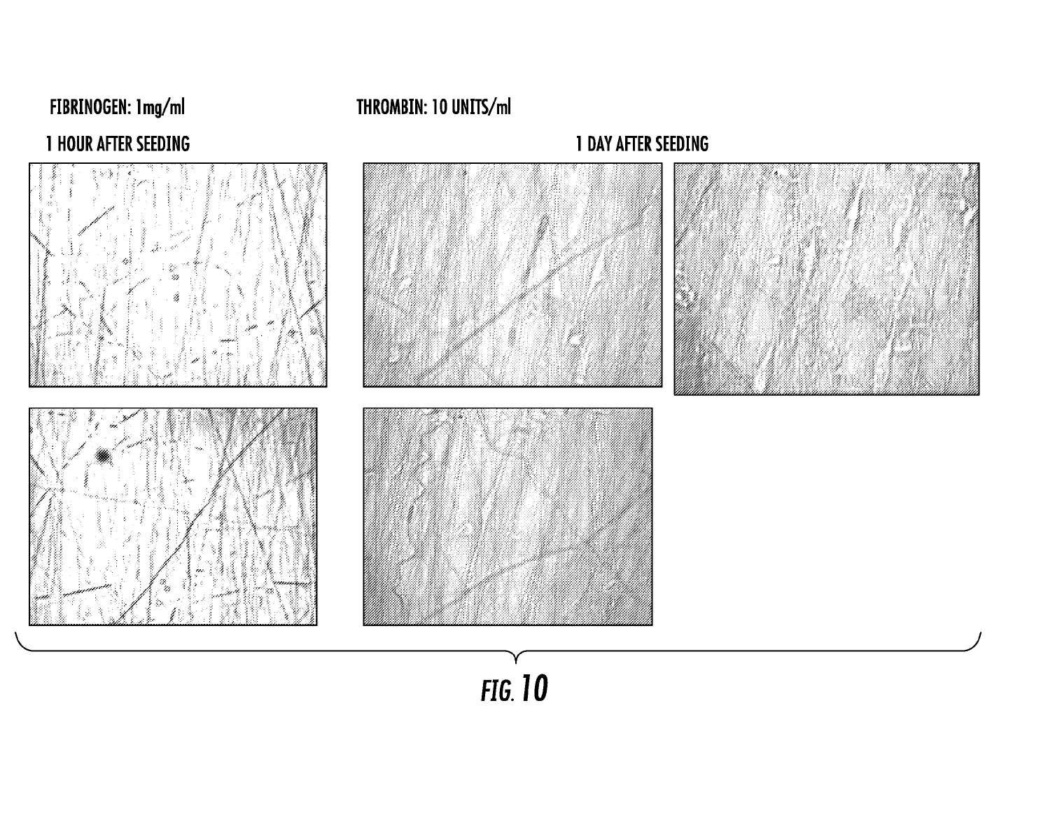

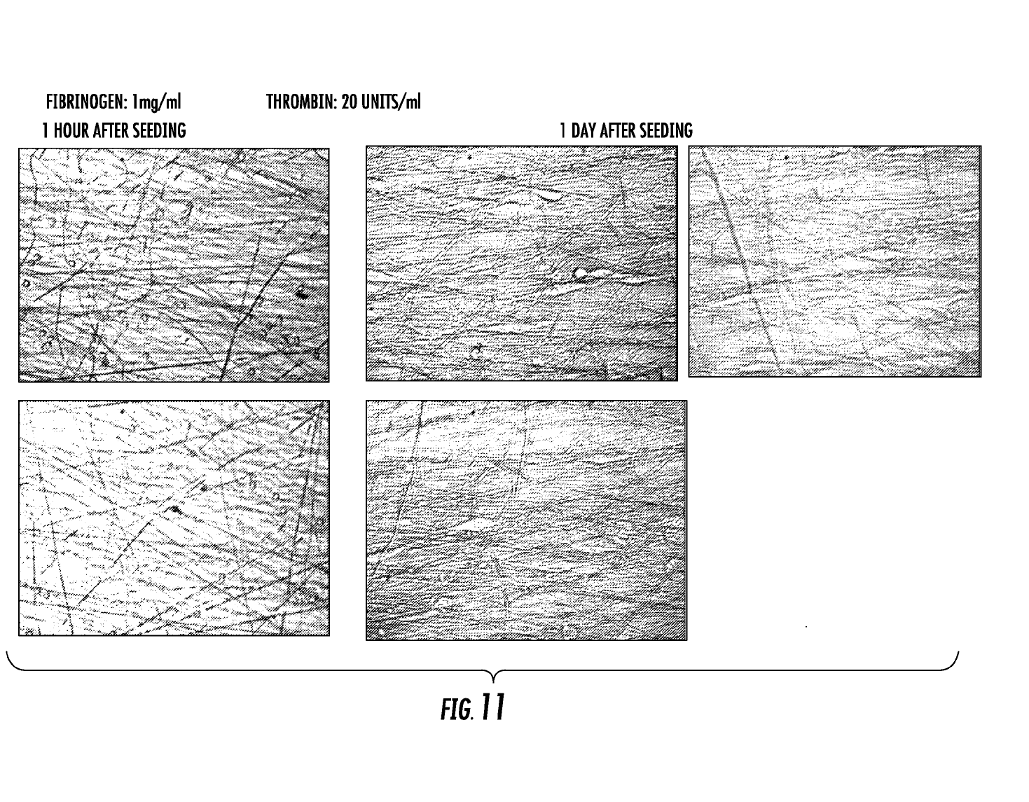

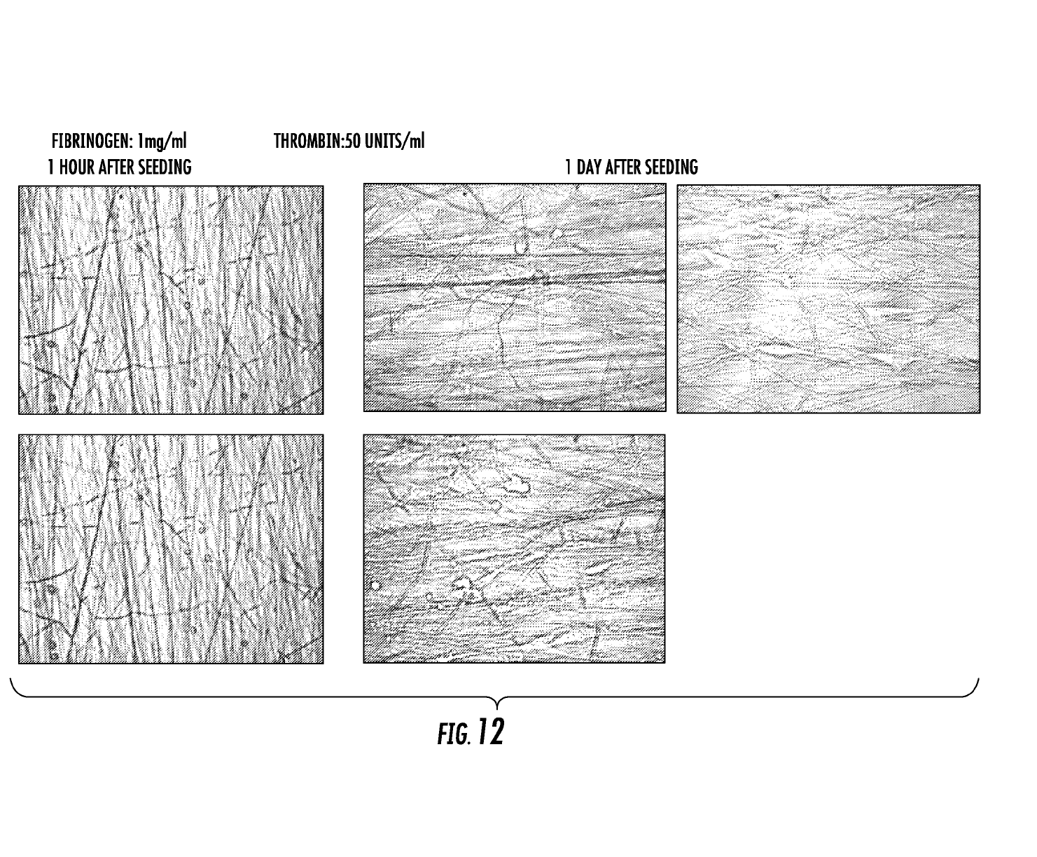

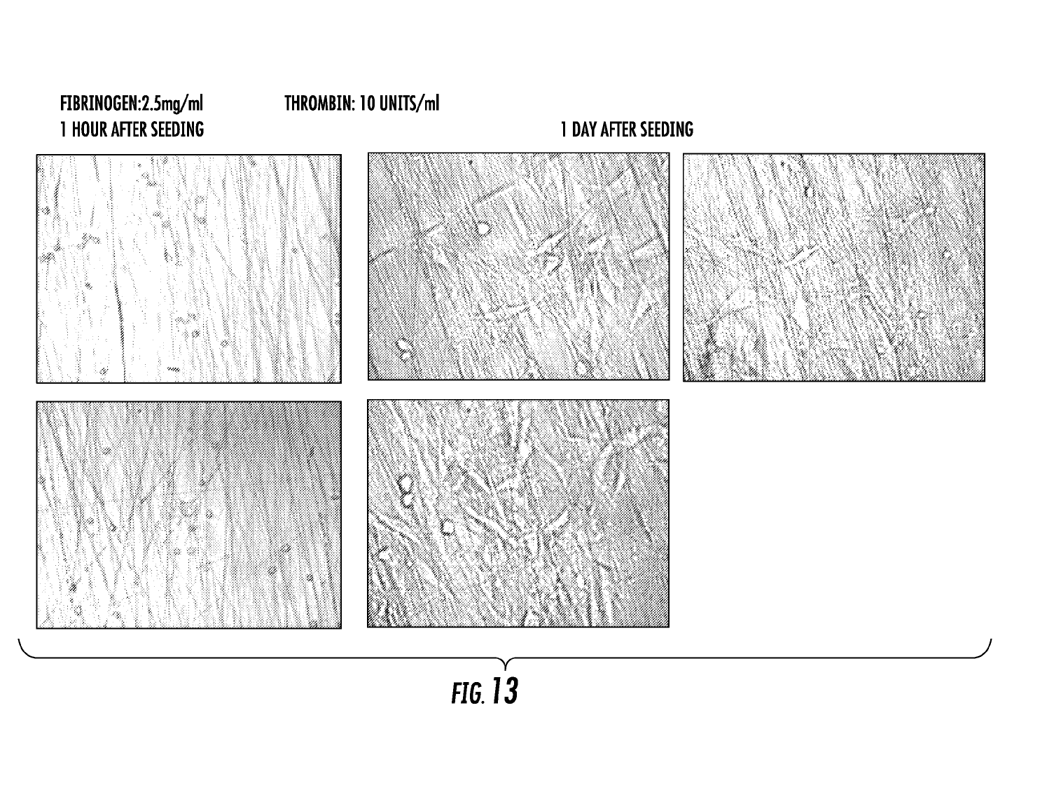

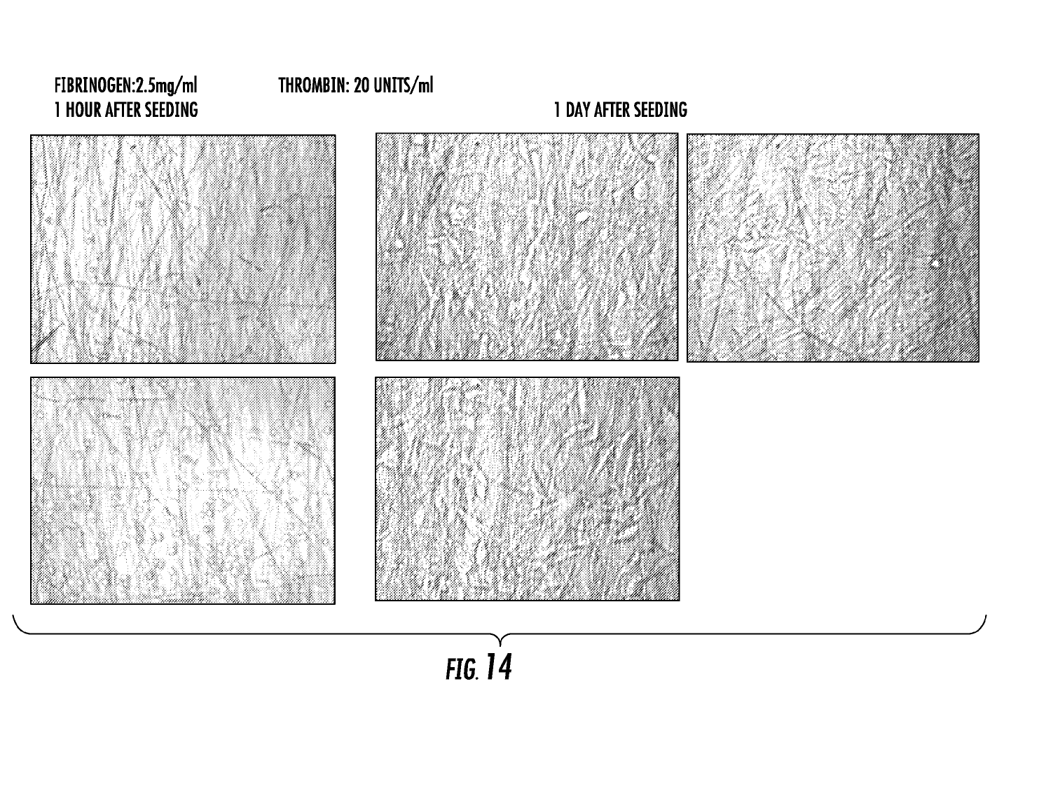

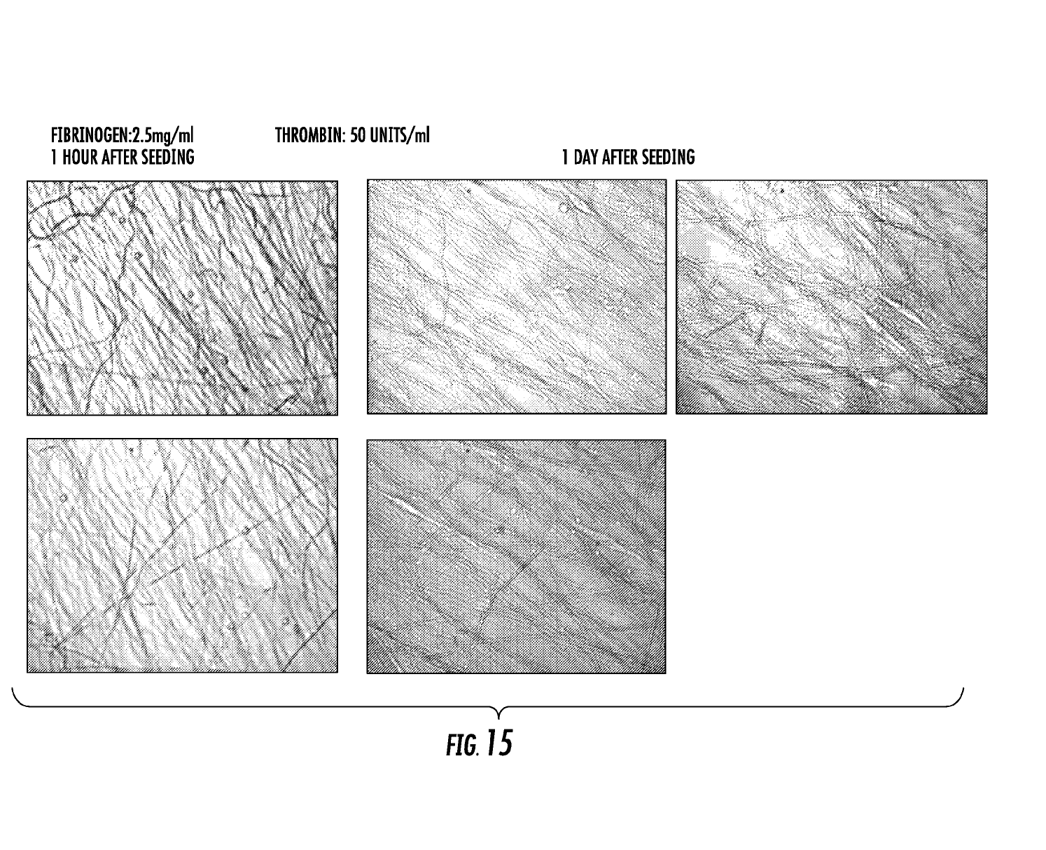

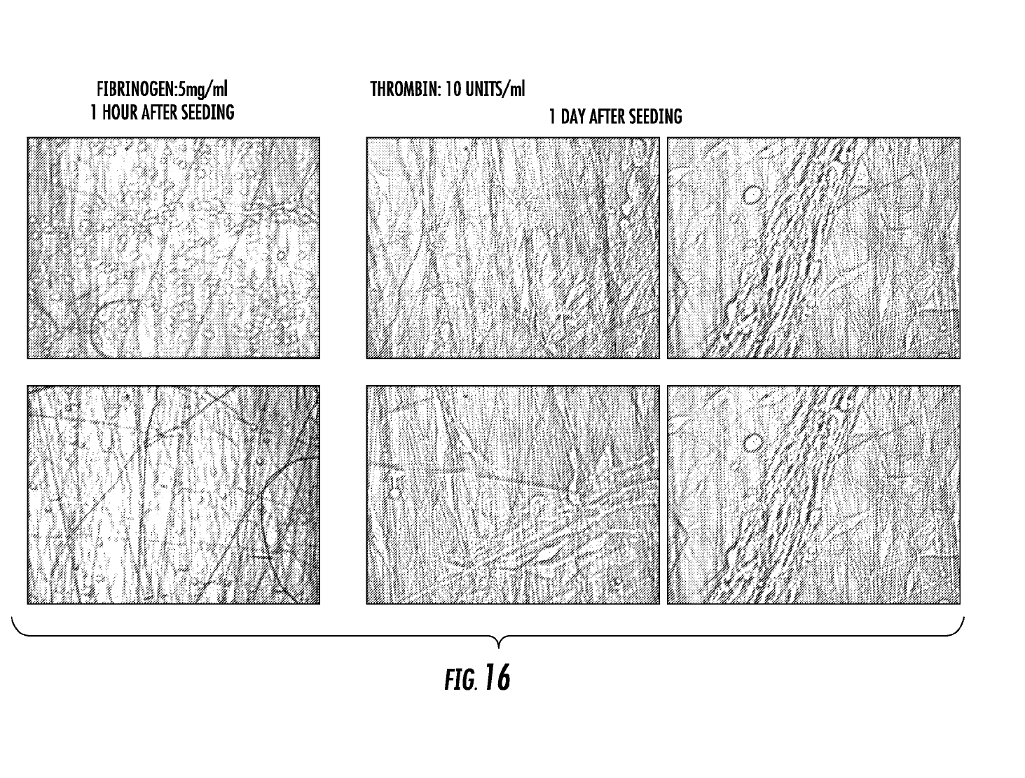

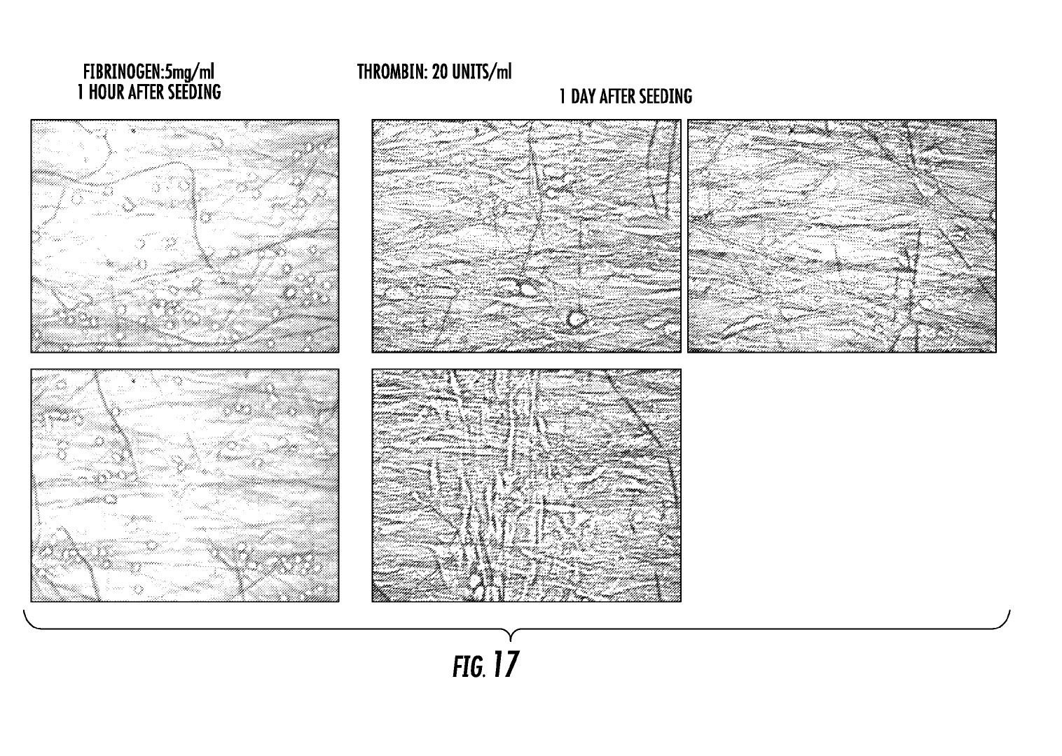

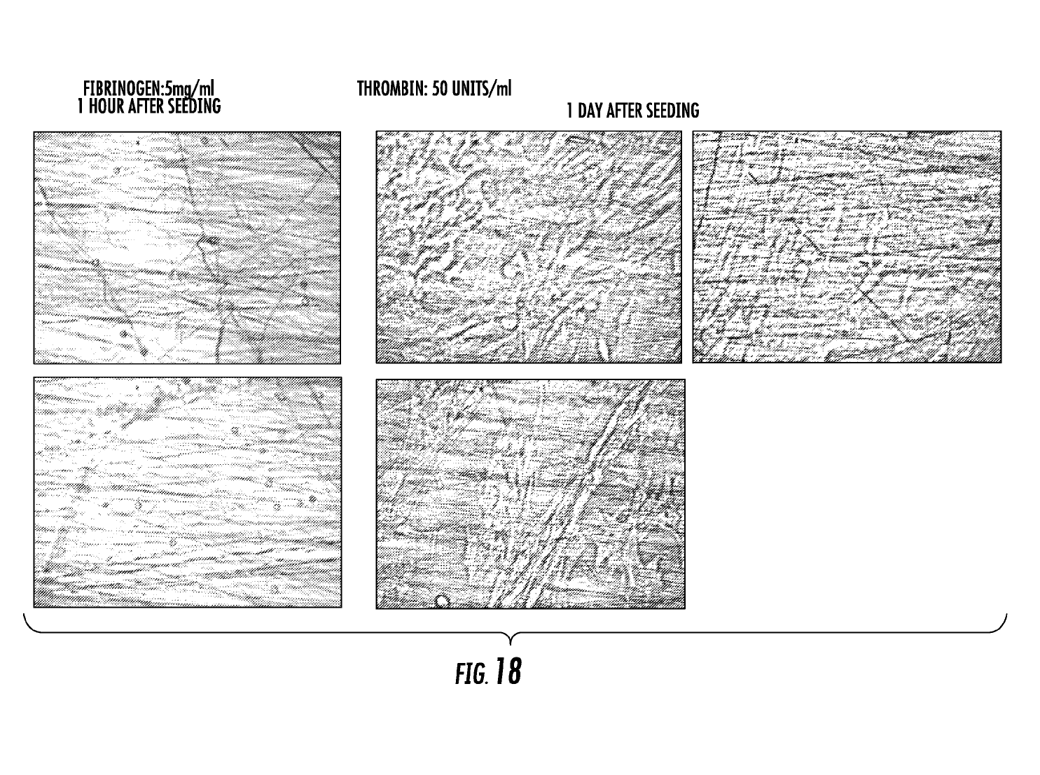

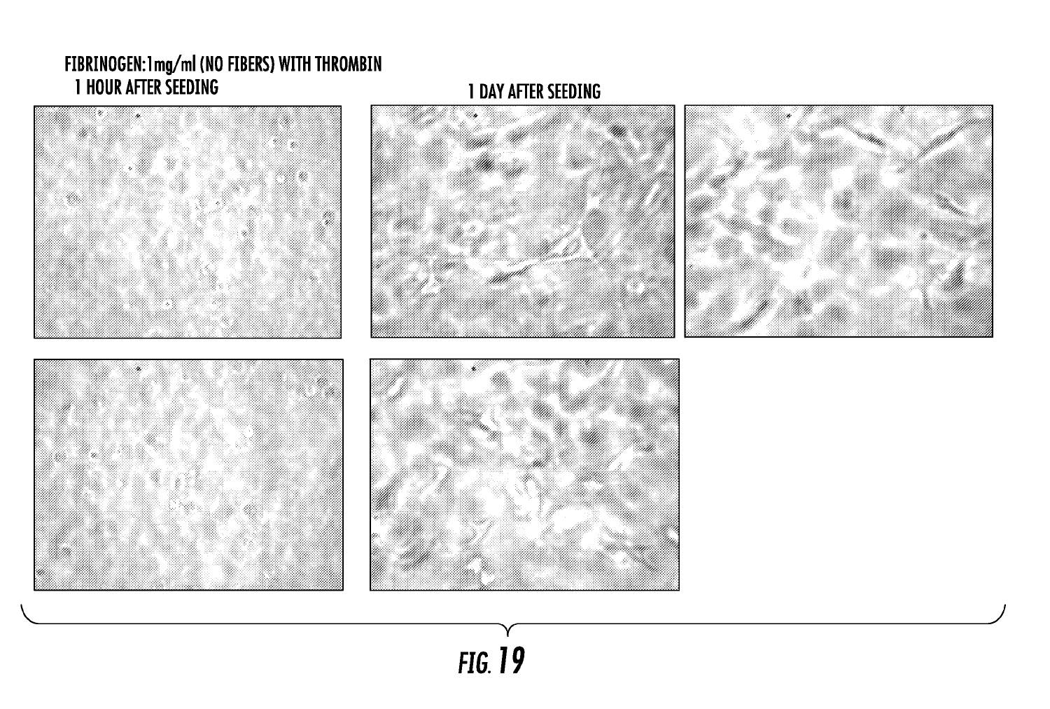

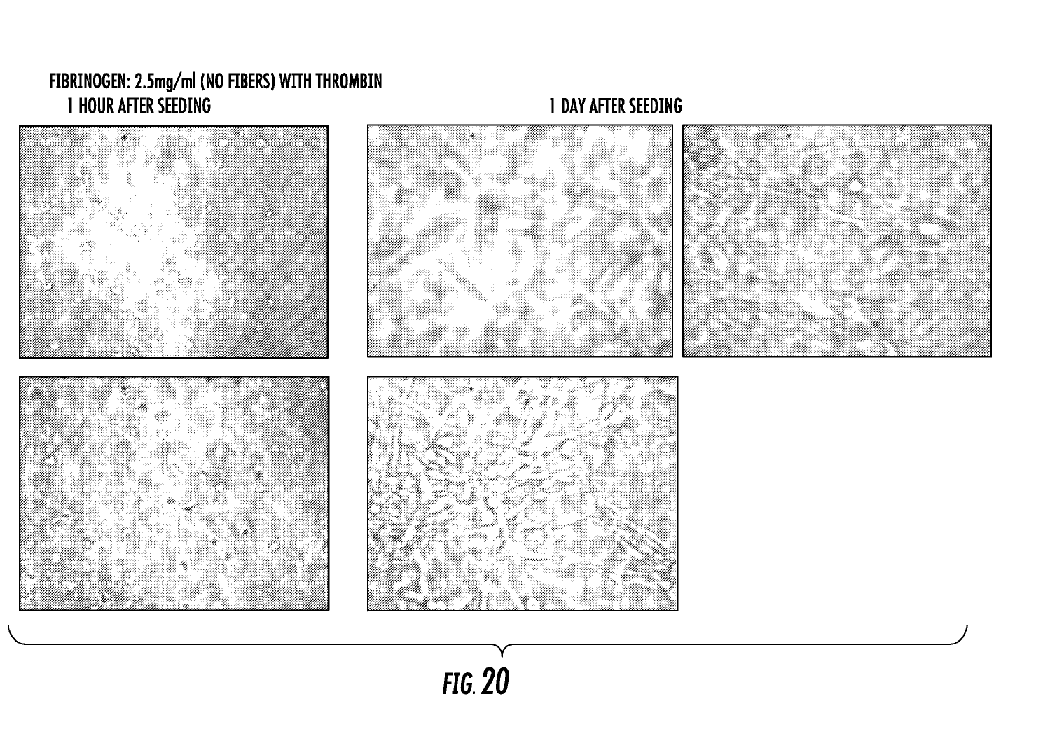

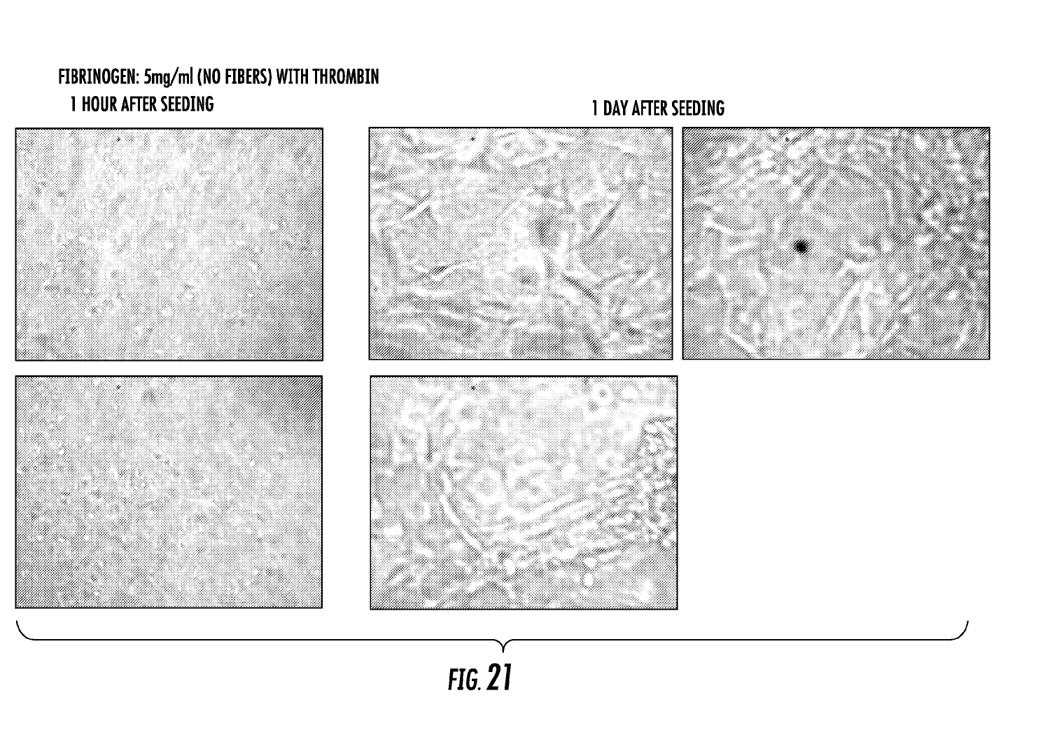

Fibroblast cells were encapsulated in the fiber/protein constructs for all nine conditions and for all three control wells without fibers. One hour after fabrication, cell morphology was rounded for all samples and controls. After one day of culture, fibroblast cells spread out and adopted an elongated morphology. It was estimated from observation that myoblast fibroblast cells proliferated and migrated in all samples and controls over 3 days of culture. FIGS. 10-21 provide several images of each of the 12 systems at both one hour after cell seeding and one day after cell seeding.

Systems were also formed including multiple layers of composite arrays, i.e., following formation of a first layer including electrospun fibers and a protein network, another layer of electrospun fibers was located on the surface of the composite and this construct was then embedded in the protein network as described previously. The procedure was repeated to form composite arrays including multiple layers.

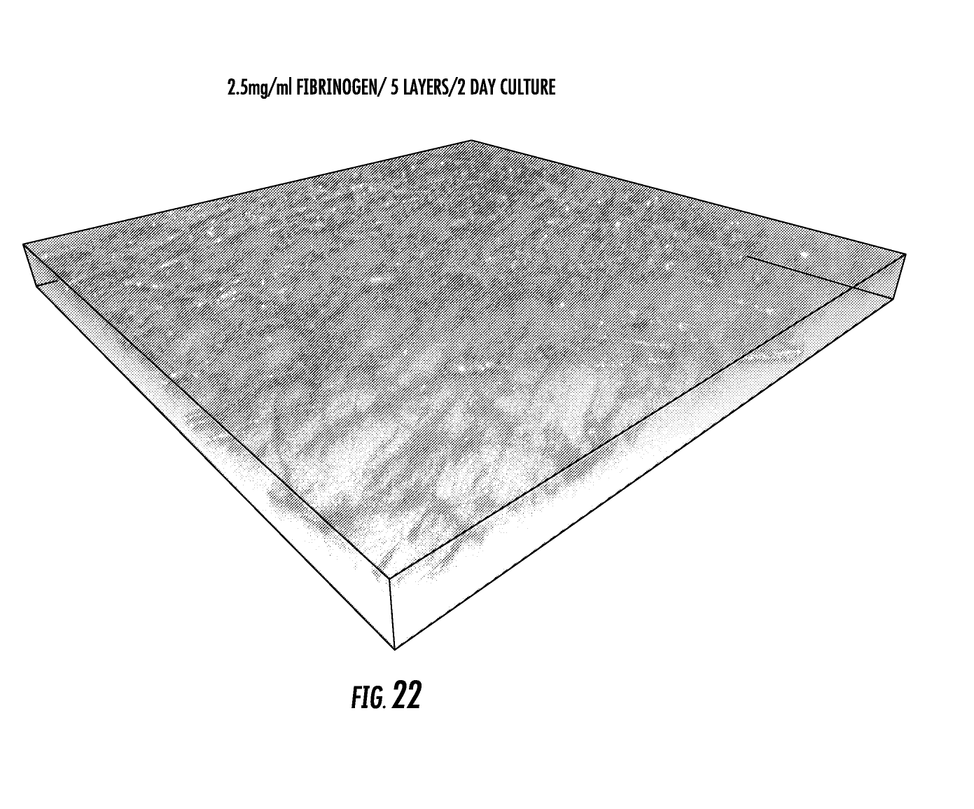

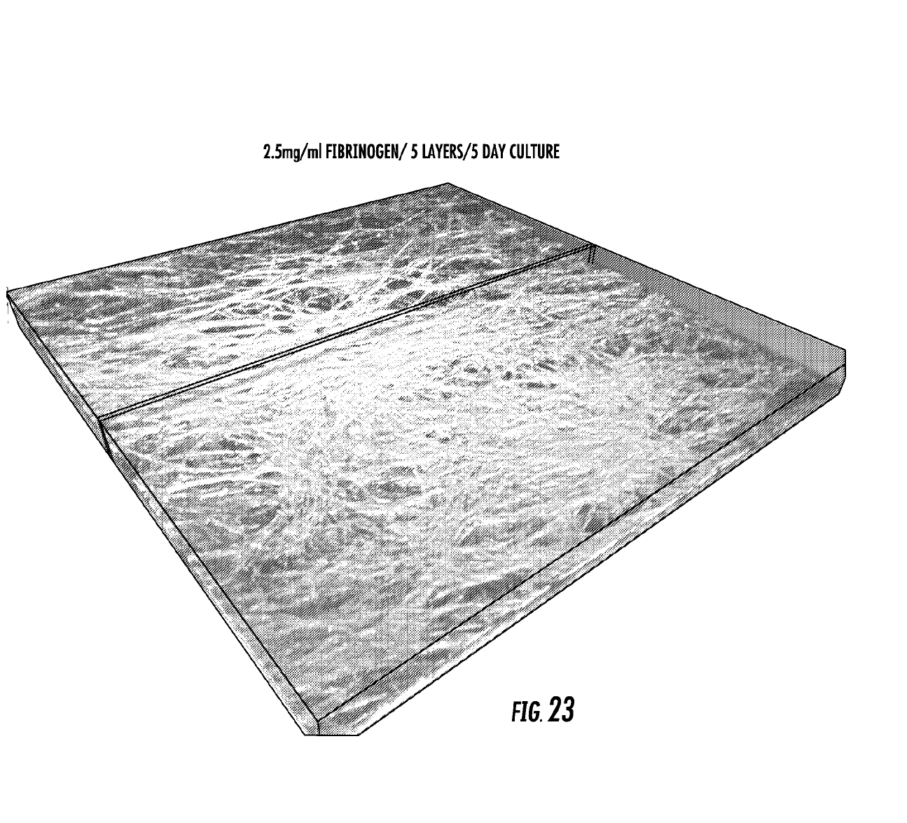

FIGS. 22 and 23 are confocal images of a five layer composite array including a protein network of 2.5 mg/ml fibrinogen. The composite array was loaded with myoblasts. The images were taken following two days of culturing.

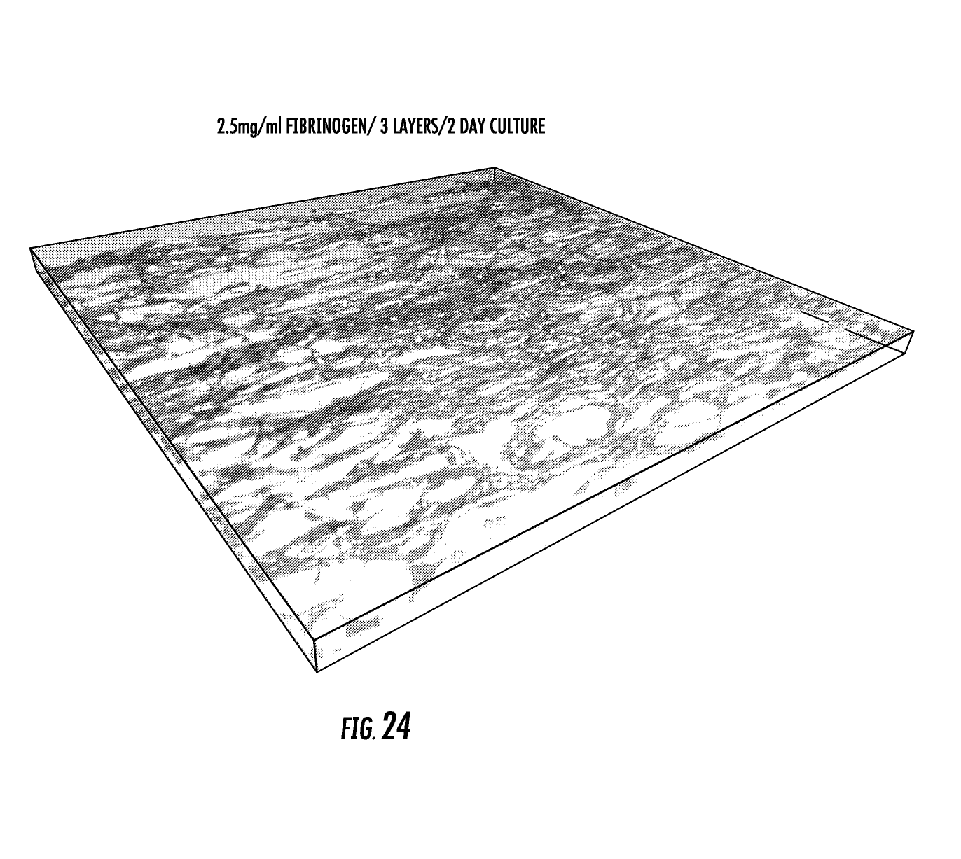

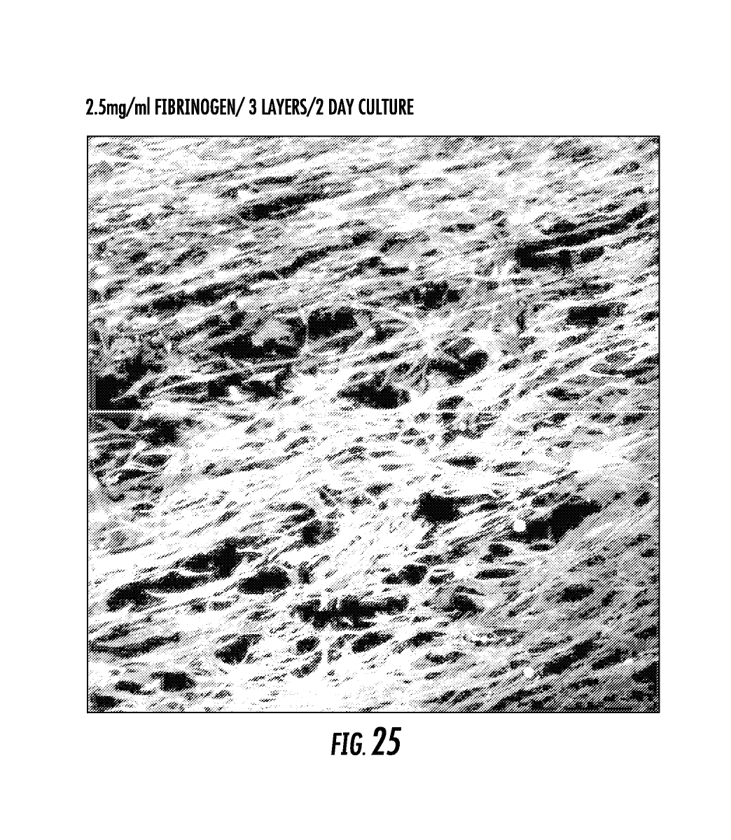

FIGS. 24 and 25 are confocal images of a three layer composite including a protein network of 2.5 mg/ml fibrinogen. The composite array was loaded with myoblasts as described above. The images were taken following two days of culturing.

### Example 3

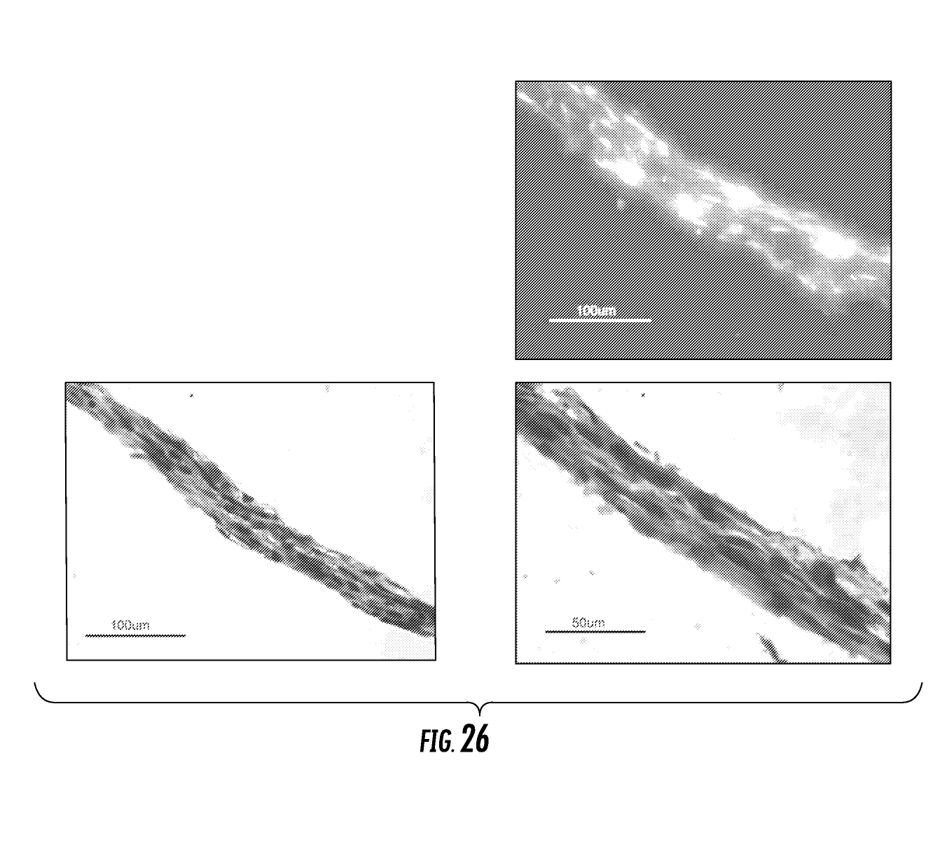

Aligned composite fiber arrays as described above in Example 1 were formed. A first array was dipped in a 15 mg/mL fibronectin solution including viable cells. A second array was dipped in a 25 unit/mL thrombin solution including 20% glycerin to increase viscosity of the solution and viable cells. Following, the two encapsulated, wet arrays were aligned adjacent to one another with the fibers of the two arrays generally aligned with one another. The two-layer composite array was allowed about 1 to about 3 minutes to crosslink. Following, three formed composite sheets were stacked together. The three layer stack was then dipped into a fibrinogen/thrombin solution and allowed between about 2 to about 3 minutes to crosslink. FIGS. 26A-C illustrate a multi-layer composite formed according to this method.

It will be appreciated that the foregoing examples, given for purposes of illustration, are not to be construed as limiting the scope of this disclosure. Although only a few exemplary embodiments have been described in detail above, those skilled in the art will readily appreciate that many modifications are possible in the exemplary embodiments without materially departing from the novel teachings and advantages of this disclosure. Accordingly, all such modifications are intended to be included within the scope of this disclosure which is herein defined and all equivalents thereto. Further, it is recognized that many embodiments may be conceived that do not achieve all of the advantages of some embodiments, yet the absence of a particular advantage shall not be construed to necessarily mean that such an embodiment is outside the scope of the present disclosure.

1. A biocompatible composite structure comprising:
a first array of biocompatible electrospun nanofibers, adjacent nanofibers of the first array being substantially aligned with one another and defining a space between one another; and
a first film encapsulating the first array of biocompatible electrospun nanofibers, the first film comprising at least one biocompatible hydrogel-forming polymer, the first film being continuous across the space between adjacent nanofibers.

2. The biocompatible composite structure according to claim 1, wherein the biocompatible hydrogel-forming polymer is an extracellular matrix protein.

3. The biocompatible composite structure according to claim 1, wherein the first array of biocompatible electrospun nanofibers comprises a polycaprolactone polymer.

4. The biocompatible composite structure according to claim 1, wherein the biocompatible composite structure is dried or hydrated.

5. The biocompatible composite structure according to claim 1, further comprising a living cell loaded onto the biocompatible composite structure.

6. The biocompatible composite structure according to claim 1, further comprising a second array of biocompatible electrospun nanofibers, adjacent fibers of the second array being substantially aligned with one another and defining a space between one another, wherein the first array and the second array are layered upon one another.

7. The biocompatible composite structure according to claim 6, wherein the first film encapsulates both the first array and the second array.

8. The biocompatible composite structure according to claim 6, wherein the first film encapsulates the first array and a second film encapsulates the second array such that the first film and the second film extend into the space between the first array and the second array, the first film and the second film being the same as or different from one another.

9. The biocompatible composite structure according to claim 6, wherein the fibers of the first array are substantially aligned with or are at an angle to the fibers of the second array.

10. The biocompatible composite structure according to claim 1, wherein the hydrogel-forming polymer of the first film is directly or indirectly bonded to another component of the composite structure.

11. A method for forming a biocompatible composite structure, the method comprising:
forming a first array of biocompatible electrospun nanofibers, adjacent nanofibers of the first array being substantially aligned with one another and defining a space between one another; and
encapsulating the first array of biocompatible electrospun nanofibers in a first film, the first film comprising at least one hydrogel-forming polymer, the first film being continuous across the space between adjacent nanofibers.

12. The method according to claim 11, wherein the hydrogel-forming polymer is an extracellular matrix protein.

13. The method according to claim 11, wherein the first array of biocompatible electrospun nanofibers is formed according to a method comprising:
electrospinning a first nanofiber from an electrospinning nozzle;
depositing the first nanofiber at a deposition area, the deposition area being defined by a first collection surface and a second collection surface, the first and second collection surfaces being separated from one another by a space, wherein a first end of the first nanofiber is adhered to the first collection surface and a second end of the first nanofiber is adhered to the second collection surface;
moving the first collection surface and the second collection surface such that the first nanofiber is moved in a direction away from the deposition area;
electrospinning a second nanofiber from the electrospinning nozzle; and
depositing the second nanofiber at the deposition area subsequent to the motion of the first nanofiber away from the deposition area.

14. The method according to claim 11, further comprising forming a second array of biocompatible electrospun nanofibers and layering the first array and the second array, adjacent fibers of the second array being substantially aligned with one another and defining a space between one another.

15. The method according to claim 14, wherein the step of encapsulating the first array of nanofibers in the first film comprises encapsulating both the first array and the second array in the first film.

16. The method according to claim 14, further comprising encapsulating the second array in a second film prior to layering the first array and the second array, the second film being the same or different as the first film.

17. The method according to claim 14, wherein the first array and the second array are layered such that the nanofibers of the first array and the nanofibers of the second array are aligned with one another or are at an angle to one another.

18. The method according to claim 11, further comprising heating a solution of the hydrogel-forming polymer prior to encapsulating the first array in the first film.

19. The method according to claim 11, further comprising drying or hydrating the film

20. The method according to claim 11, further comprising loading a living cell onto the biocompatible composite structure.

21. The method according to claim 11, further comprising bonding the hydrogel-forming polymer of the first film to another component of the biocompatible composite structure, the hydrogel-forming polymer of the first film being bonded to another polymer that is either the same or different as the hydrogel-forming polymer, a non-polymeric component of the biocompatible composite structure, or a nanofiber.
